(19) United States
(12) Patent Application Publication
(10) Pub. No.: US 2006/0020167 A1
(43) Pub. Date: Jan. 26, 2006

(54) Medical devices for minimally invasive surgeries and other internal procedures

(76) Inventor: James Sitzmann, Zionsville, IN (US)

Correspondence Address: DAVID G. HENRY, 900 Washington Ave., P.O. Box 1470, Waco, TX, 76703-1470, US

(21) Appl. No.: 11/172,385

(22) Filed: Jun. 30, 2005

Related U.S. Application Data
(60) Provisional application No. 60/583,720, filed on Jun. 30, 2004.

(51) Int. Cl. A61B 17/00; A61B 1/06

(52) U.S. Cl. 600/173; 600/182; 600/166; 606/1

(57) ABSTRACT

Minimally invasive surgical devices and related tools are provided. The devices include image acquisition devices and forceps, scissors, clamps, ultrasound probes, lasers, cautery devices, staplers, knives, suturing devices, rivet drivers, ligation devices, aspiration devices, injection devices, biopsy devices, radiotherapy devices; and radioactive emitter loading devices. Other devices useful for internal procedures in a patient's body or for facilitating such procedures are also provided.

### FIELD OF THE INVENTION

In certain embodiments, the present invention relates to medical devices for internal use in a patient's body and, in particular, devices related to minimally invasive surgeries and other medical procedures.

### BACKGROUND OF THE INVENTION

Various tools and procedures are known for use in minimally-invasive surgery. These tools and procedures are favored over “open” surgical techniques as the minimally-invasive techniques reduce patient discomfort and facilitate rapid healing and recovery. “Open” surgical techniques typically require the use of large incisions to gain entry to the interior of the body. “Open” surgeries also typically require a longer post-operative hospital stay and cause increased post-operative pain. The large incisions of “open” surgeries may leave large and sometimes unsightly scars.

Minimally-invasive surgical procedures, on the other hand, may often be conducted on an outpatient basis. Minimally invasive surgeries are often performed with relatively small surgical incisions or ports which, in contrast with the large incisions of “open surgery” have a decreased risk of infection. Minimally invasive surgery is also desirable for the benefit of generally avoiding much of the internal damage resulting from the larger incisions of “open” surgeries, such as the cutting of abdominal muscle and other tissue required to gain access to the abdominal body cavity in an “open” surgery. Because it is not as disruptive as “open surgery”, minimally invasive surgery may be used as a diagnostic tool, enabling a physician to visually inspect, and even sample, certain tissues.

The presently available devices for minimally invasive surgical procedures all have certain inherent disadvantages, including, without limitation, difficulty and/or discomfort in use of the devices, limited features, and sensory and sensitivity loss between the operator and the material being examined or manipulated. The presently available devices are also somewhat difficult to use due to the limited vision provided by the cameras that are available for a surgeon to see his or her work.

These disadvantages combine to sometimes render minimally invasive surgical procedures more difficult than is desirable. The difficulty of the procedures, the lack of tactile perception, and the limited working area may increase the likelihood of accidental damage to the organs, vessels, and other tissues surrounding the surgical area.

A need exists for minimally invasive surgical devices that provide a surgeon with a wider variety of options than is presently available. Further, it is desirable to provide minimally invasive surgical devices that do not include the disadvantages of the presently-available devices.

### SUMMARY OF THE INVENTION

One object of the present invention is to provide new devices useful for minimally invasive surgeries and other procedures which are performed within a patient's body that allow a surgeon to minimize unnecessary damage to the organs, vessels, and other tissues surrounding the surgical area.

A further object of the invention is to provide devices which facilitate an expanded field of vision during minimally invasive surgeries and other internal procedures.

Another object of the invention is to provide devices which facilitate easier handling and manipulation of instruments for minimally invasive surgeries and other internal procedures.

Another object of the invention is to provide devices for minimally invasive surgeries and other internal procedures which facilitate increased tactile and/or visual perception for the surgeon.

Yet another object of the invention is to provide devices for minimally invasive surgeries and other internal procedures which expand the range of options available to a surgeon.

According to one embodiment of the invention, an optical device is provided for minimally invasive medical procedures that facilitates stereoimagery through the use of multiple image acquisition devices. The optical device includes a plurality of linear image acquisition devices, and at least two of the linear image acquisition devices are adapted to receive an image from within a patient's body. The optical device also includes a linear housing laterally surrounding the linear image acquisition devices so that the linear image acquisition devices extend toward one end of the housing. The housing is such that at least a portion of it may be inserted within a patient's body and the housing is also adapted for cleaning. At least one input adjustment device is disposed upon at least one end of the plurality of linear image acquisition devices and the input adjustment device may include a lens and/or a reflective surface.

According to another embodiment of the invention, a medical device is provided with a hand piece, an instrument portion including a tool, and one or more control elements. The control elements are useful to operate or manipulate features of the device. One of the control elements may be a trigger. The hand piece may be configured for a right hand of a user, or a left hand of a user, or for use by either hand of a user.

According to another embodiment of the invention, a device for minimally invasive medical procedures is provided. The device includes a scissor-type hand piece with a first elongated portion and a second elongated portion. The first elongated portion is adapted for manipulation by a user's thumb and the second elongated portion is adapted for manipulation by one or more of a user's first, second, third, and fourth fingers. A temperature control element may also be provided to generate a signal upon manipulation of the control element. A tool is also provided, and is responsive to the signal generated by the temperature control element. In response to the signal of the temperature control element, at least a portion of the tool heats up, so as to be useful to cauterize tissue. Optionally, the tool may be detachable from the device.

According to another embodiment of the invention, a tool for use in minimally invasive medical procedures is provided. The tool includes an elongated first element, an elongated second element, and an elongated third element. The first and second element are opposed to the third element. The first element is configured to mimic the functionality of a first finger of a user, and the second element is configured to mimic the functionality of a second finger of a user. The third element is configured to mimic the functionality of a third finger of a user. Each of the first element, second element, and third element are configured to transmit a pressure sensation from that element to a user's finger.

According to another embodiment of the invention, an automated device for minimally invasive medical procedures, is provided. The device includes a robotic console, a plurality of control features, and one or more robotic limbs.

### BRIEF DESCRIPTION OF THE DRAWINGS

FIG. 1a shows a cross-sectional view of an optical device in accordance with one embodiment of the invention;

FIG. 1b shows a cross-sectional view of an optical device in accordance with another embodiment of the invention;

FIG. 2a depicts a portion of an optical device in accordance with an embodiment of the present invention;

FIG. 2b depicts a portion of an optical device in accordance with another embodiment of the present invention;

FIG. 2c depicts a portion of an optical device in accordance with another embodiment of the present invention;

FIG. 3 shows a block diagram of a system according to an embodiment of the invention;

FIG. 4a shows a lateral view of certain components of an optical device in accordance with an embodiment of the invention;

FIG. 4b shows a cross-sectional view of an optical device in accordance with one embodiment of the invention;

FIG. 4c shows a top view of an optical device in accordance with one embodiment of the invention;

FIGS. 4d, 4e, 4f, 4g, 4h, 4i, 4j and 4k show a lateral views of a portion of an optical device in accordance with an embodiment of the invention;

FIG. 5a shows a lateral view of certain components of a medical device in accordance with an embodiment of the invention;

FIG. 5b shows a lateral view of certain components of a medical device in accordance with an embodiment of the invention;

FIG. 5c shows a schematic diagram of certain components of a medical device in accordance with an embodiment of the invention;

FIG. 6 shows a lateral view of certain components of a medical device in accordance with an embodiment of the invention;

FIG. 7a shows a lateral view of a medical device in accordance with an embodiment of the invention;

FIG. 7b shows a lateral view of the medical device of FIG. 7a in a different position;

FIG. 8 shows a lateral view of a medical device in accordance with an embodiment of the invention;

FIG. 9 shows a lateral view of a medical device in accordance with an embodiment of the invention;

FIGS. 10a, 10b, 10c, 10d, and 10e show various lateral views of components for use as part of medical devices in accordance with certain embodiments of the invention;

FIGS. 11a, 11b, 11c, and 11d show lateral views of components for use as part of a medical device in accordance with certain embodiments of the invention;

FIGS. 12a, 12b and 12c show lateral views of tools for use as part of a medical device in accordance with certain embodiments of the invention;

FIG. 13a shows a lateral view of an ultrasonic device in accordance with one embodiment of the invention;

FIG. 13b shows a screen image for use with the ultrasonic device of FIG. 13a, in accordance with one embodiment of the invention;

FIG. 13c shows two ultrasonic probes for use with an embodiment of the invention;

FIG. 14a shows an laser in accordance with an embodiment of the invention;

FIG. 14b shows a screen image for use with the laser device of FIG. 14a, in accordance with an embodiment of the invention;

FIG. 15a shows a lateral view of a medical device in accordance with an embodiment of the invention;

FIG. 15b shows a lateral view of another medical device in accordance with an embodiment of the invention;

FIG. 16 depicts a variety of components for use with certain medical devices of the present invention;

FIG. 17 shows a variety of different cautery devices for use in accordance with certain embodiments of the invention;

FIGS. 18a and 18b depict a device for use in accordance with certain embodiments of the invention;

FIGS. 19a, 19b and 19c depict another device for use in accordance with certain embodiments of the invention;

FIGS. 20a and 20b depict arrangements for stapling tissue in accordance with certain embodiments of the invention;

FIG. 21a depicts a suturing device in accordance with an embodiment of the invention;

FIG. 21b depicts a portion of the suturing device of FIG. 21a;

FIGS. 21c and 21d depict a lateral view of a suturing device in accordance with an embodiment of the invention;

FIGS. 21e, 21f and 21g depict views of a portion of another suturing device in accordance with an embodiment of the invention;

FIG. 21h depicts a lateral view of a suturing device in accordance with an embodiment of the invention;

FIGS. 21i and 21j depict views of a portion of the suturing device of FIG. 21h;

FIGS. 21k and 21l depict cross-sectional views of a portion of the suturing device of FIG. 21h;

FIGS. 21m, 21n, 21o, 21p, 21q, 21r, and 21s depict lateral views of various portions of devices in accordance with certain embodiments of the invention;

FIG. 22a depicts a bobbit-style suture holder device for use in accordance with certain embodiments of the invention;

FIG. 22b depicts a base for use in accordance with a suturing device such as that depicted in FIG. 21a, and a bobbit assembly such as that depicted in FIG. 22a;

FIG. 22c depicts an arrangement similar to that shown in FIG. 22a, with an external bobbit assembly;

FIG. 22d depicts a suture catcher for use in accordance with certain embodiments of the invention;

FIGS. 22e, 22f, 22g, 22h, 22i, 22j, 22k, 221, 22m, 22n, 22p, 22o, 22q, 22r, 22t, 22s, 22u and 22v depict views of portions of a suturing device in accordance with an embodiment of the invention;

FIG. 23a depicts a rivet driver in accordance with another embodiment of the invention;

FIG. 23b depicts the components of a fastener in accordance with an embodiment of the invention;

FIG. 23c depicts another view of the components of a fastener, such as that shown in FIG. 23b;

FIG. 23d depicts another view of the components of a fastener, such as that shown in FIG. 23b;

FIG. 23e depicts another view of the components of a fastener, such as that shown in FIG. 23b;

FIG. 23f depicts a cartridge for a group of fasteners such as the fastener shown in FIG. 23b;

FIGS. 23g, 23h, 23i, and 23j depict views of various portions of fastening devices in accordance with certain embodiments of the invention;

FIG. 24a depicts a needle driver for use in accordance with certain embodiments of the invention;

FIG. 24b depicts a needle similar to that shown in FIG. 24a, with the bevel lock in an open position;

FIG. 24c depicts a needle similar to that shown in FIG. 24a, however the needle of FIG. 24c has an inverted bevel;

FIG. 24d depicts the needle of FIG. 24c, with the bevel lock in an open position;

FIG. 24e depicts various needles for use in accordance with certain embodiments of the invention;

FIG. 25a depicts various needles for use in accordance with certain embodiments of the invention;

FIGS. 25b, 25c, 25d, 25e, 25f and 25g depicts cross-sectional views of certain needles for use in accordance with certain embodiments of the invention;

FIG. 26 depicts a pistol-style ligation device in accordance with certain embodiments of the invention;

FIG. 27a depicts one embodiment of a grasping rod for use in accordance with one embodiment of the invention;

FIG. 27b depicts another embodiment of a manipulation or grasping rod for use in accordance with an embodiment of the invention;

FIG. 27c depicts a manipulation or grasping rod for use in accordance with an embodiment of the invention;

FIG. 27d depicts an adhesive ligation staple rod for use in accordance with an embodiment of the invention;

FIG. 27e depicts an adhesive stapler loading device in accordance with an embodiment of the invention;

FIG. 27f depicts an injector which injects an adhesive substance into a compressor mold in accordance with one embodiment of the invention;

FIG. 27g depicts an injector similar to that shown in FIG. 27f;

FIG. 28a depicts a fastener for use in accordance with an embodiment of the invention;

FIG. 28b depicts the fastener of FIG. 28a in a closed position;

FIG. 28c depicts a view of a fastener similar to that shown in FIG. 28a, in a different position;

FIG. 29a depicts a fastener similar to that depicted in FIG. 28a for use in accordance with an embodiment of the invention;

FIG. 29b depicts the fastener of FIG. 29a in a closed position;

FIG. 29c depicts the fastener FIG. 29a in another closed position;

FIG. 30a depicts a circular stapler device in accordance with an embodiment of the invention;

FIG. 30b shows the circular stapler device of FIG. 30a in another position;

FIGS. 31a, 31b, and 31c depict portions of a circular stapler similar to the circular stapler shown in FIGS. 30a and 30b.

FIGS. 32a and 32b depict views of a circular staple head;

FIG. 33 depicts a portion of a circular stapler in accordance with an embodiment of the invention;

FIG. 34 depicts a side view of a portion of a circular stapler in accordance with an embodiment of the invention;

FIG. 35 depicts a side view of a surgical device in accordance with an embodiment of the invention;

FIG. 36a depicts a side view of a surgical device similar to that shown in FIG. 35;

FIG. 36b depicts a side view of a portion of the surgical device of FIG. 36a;

FIG. 37a depicts a portion of a medical device in accordance with an embodiment of the invention;

FIGS. 37b, 37c, 37d, 37e, and 37f depict views of different embodiments of a components for use with a medical device similar to that shown in FIG. 37a;

FIG. 38 shows a side view of a portion of a medical device for use in accordance with an embodiment of the invention;

FIG. 39 shows a side view of a medical device in accordance with another embodiment of the invention;

FIG. 40a depicts a view of a medical device in accordance with another embodiment of the invention;

FIG. 40b depicts a view of a portion of a medical device for use in accordance with a device similar to that shown in FIG. 40a;

FIGS. 40c and 40d depict a view of a portion of a medical device for use in accordance with a device similar to that shown in FIG. 40b;

FIG. 40e depicts a view of a device for use in accordance with a medical device similar to that shown in FIG. 40b;

FIG. 41 depicts a view of a device similar to that shown in FIG. 40a, in accordance with an embodiment of the invention;

FIG. 42 depicts a view of a device for use in accordance a medical device similar to that shown in FIG. 41, in accordance with another embodiment of the invention.

### DETAILED DESCRIPTION OF PREFERRED EMBODIMENTS

The present invention may be understood by reference to the following detailed description of particular embodiments of the invention. The terminology used herein is for the purpose of describing particular embodiments only and is not intended to be limiting.

In one embodiment, the present invention provides an optical device for minimally invasive surgery that allows the user to view multiple images in a variety of arrangements or formats. The device achieves this by using multiple image acquisition devices. Each image acquisition device obtains an image and transmits it to a display device capable of displaying the images in multiple formats. In one embodiment, the device allows a user to switch between binocular and trinocular image presentation.

In one embodiment, an optical device is provided with two image acquisition devices. FIG. 1a presents a cross-sectional view of such an optical device 2. A housing 4 laterally surrounds two linear image acquisition devices 6a and 6b. The housing 4 extends linearly along the image acquisition devices 30a and 6b. At least a portion of the housing 4 is configured so that it may be inserted within a patient's body. Similarly, at least a portion of the housing 4 may be adapted for cleaning and sterilization so that the optical device 2 may be cleaned, sterilized, and reused.

The linear image acquisition devices 6a and 6b may be configured in accordance with any suitable means for image acquisition. For instance, the image acquisition devices 6a and 6b may be fiber optic cables or cameras which digitally capture an image and transmit it electronically. In the case of the later embodiment, the cameras are positioned at one end of the linear housing 4 with wiring or some other suitable means for transmission of an electronic signal extending through the linear housing 4.

Image information may be transmitted with wireless transmitters: (electronic or optical). Electronic data is preferably set to a specific wavelength and frequency to keep it secure and avoid interference with other equipment, for instance nearby medical equipment such as electromagnetic imaging equipment. A short range optic “wireless” transmission may also be used, especially where wireless electronic transmission is or could be problematic.

FIG. 1b shows a cross-sectional view of another embodiment of an optical device wherein three image acquisition devices 6a, 6b, and 6c are protected by a housing 4, which laterally surrounds the linear image acquisition devices 6a, 6b and 6c. This embodiment allows the optical device to collect three images for presentation to the user. In another embodiment (not shown) one or more light sources may be provided to provide lighting for the image acquisition devices 6a, 6b, and 6c. Alternatively, any of the image acquisition devices, for instance 6b, may be replaced with a light source to provide illumination for image acquisition devices 6a and 6c.

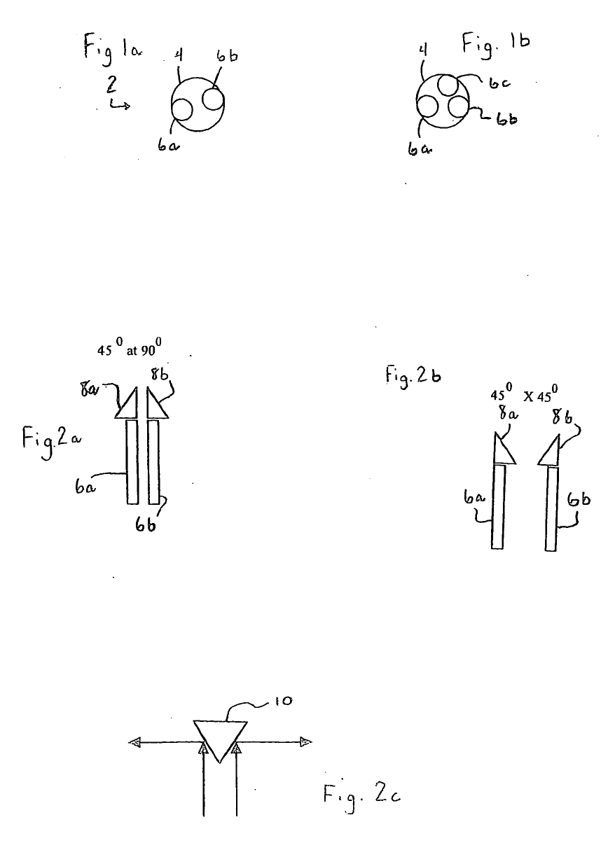

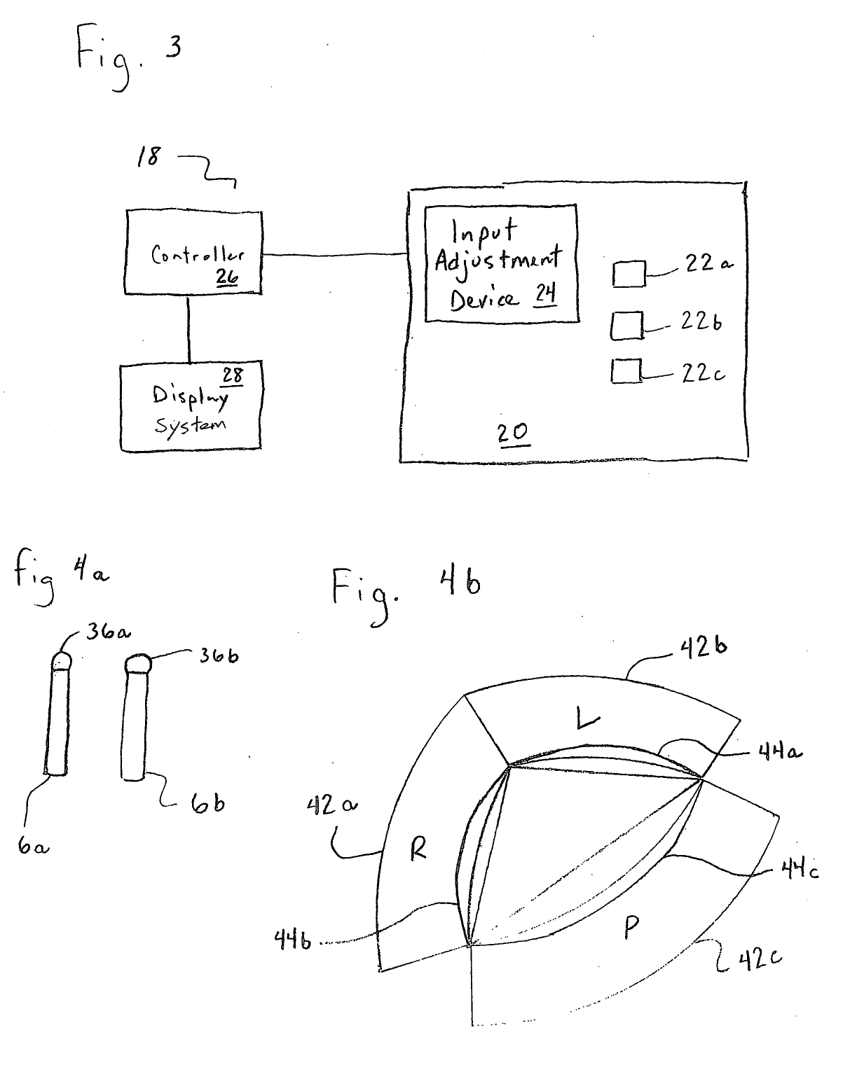

FIG. 2a depicts a view of a portion of an optical device such as that shown in FIG. 1a. In the portion of the optical device shown in FIG. 2a, two image acquisition devices 6a and 6b are provided. Each image acquisition device 6a and 6b is provided with an image adjustment device 8a and 8b. The image adjustment device 8a and 8b may be any device suitable for manipulating an image, such as a prism, a lens, a reflecting mirror, or a combination of the foregoing.

FIG. 2b depicts a view of a portion of an optical device similar to that depicted in FIG. 2a. The portion of the optical device shown in FIG. 2b includes two image acquisition devices 6a and 6b. The image adjustment devices 8a and 8b are similar, to those shown in FIG. 2a, however in FIG. 2b, the image adjustment devices 8a and 8b are oriented differently so as to provide a different field of view for each of the image adjustment devices 8a and 8b.

FIG. 2c depicts another view of a portion of an optical device similar to that depicted in FIGS. 2a and 2b, however in FIG. 2c, a single image adjustment device 10 is provided. This single image adjustment device 10 may be oriented so as to provide two images. One of each of the images is provided to a separate image acquisition device (not shown).

FIG. 3 depicts a system diagram of one embodiment of an imaging system 18 in accordance with the present invention. In this embodiment, a optical device 20 consists of a plurality of linear image acquisition devices 22a, 22b, and 22c and at least one input adjustment device 24. A controller 26 is provided for receiving and responding to instructions to control the imaging system 18. In particular, the controller 26 may be in communication with the input adjustment device 24 and the display system 28. The controller 26 may control any number of a variety of features related to image acquisition, for instance, focusing the image or rotating a mirror disposed on the image acquisition devices so as to rotate or change the view displayed by the imaging system 18. Additionally, or alternatively, the controller 26 may operate to control the display system 28. In such an embodiment, the controller 26 may control a variety of different features associated with a display device, for instance, the tint, color, brightness, sharpness, contrast or any of a variety of other features known to be adjustable in image acquisition devices. Further, the controller 26 may direct what display format the images acquired by the optical device 20 are displayed in. For instance, where two image acquisition devices (not shown) are used, the image may be displayed in either a single image or a left/right image format as two images beside each other. Where three image acquisition devices 22a-c are utilized, the third image may be presented as a screen insert. In yet another alternative where multiple image acquisition devices are used, images from any one or combination of the image acquisition devices may be selected for presentation on the display system 28.

The display system 28 may be either a traditional television style display device with a monitor and a screen, a goggle style display device with an eyeglass-type frame that presents a separate image to each eye of the user, or a combination of one or more of these. In the latter case, the images may vary between the eyes so that the left eye receives a left image provided one linear image acquisition device and the right eye receives a right image obtained by a different linear image acquisition device. Alternatively, a third image may be presented in one or both of the left eye and right eye image as an insert or, where desired by the user, as a complete replacement for either the left or right eye. Any of these options may be selected by the user through the operation of the controller 26. Where the display system 28 includes an eyeglass-frame type display device, it may optionally be configured so that the display screen of one or both eyes may be slipped up or down so as to be moved in front of entirely clear of one or both eyes of the user. In one embodiment, multiple display devices are provided.

In one embodiment, the controller 26 operates through voice activation so that the user need only verbally audibilize commands directing the controller 26 to perform some function or change in the operation of the imaging system 18. Alternatively, or in combination with voice operation, the controller 26 may provide features for manual manipulation by the user, including various control elements such as buttons, switches, dials, or any other control element which allow a user to control one or more functions of the imaging system 18.

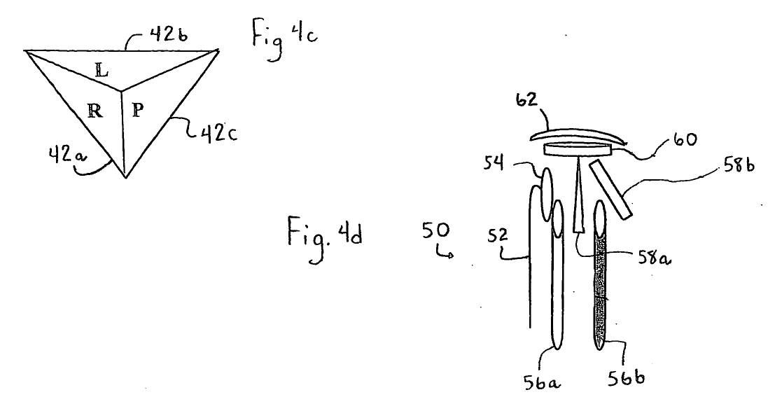

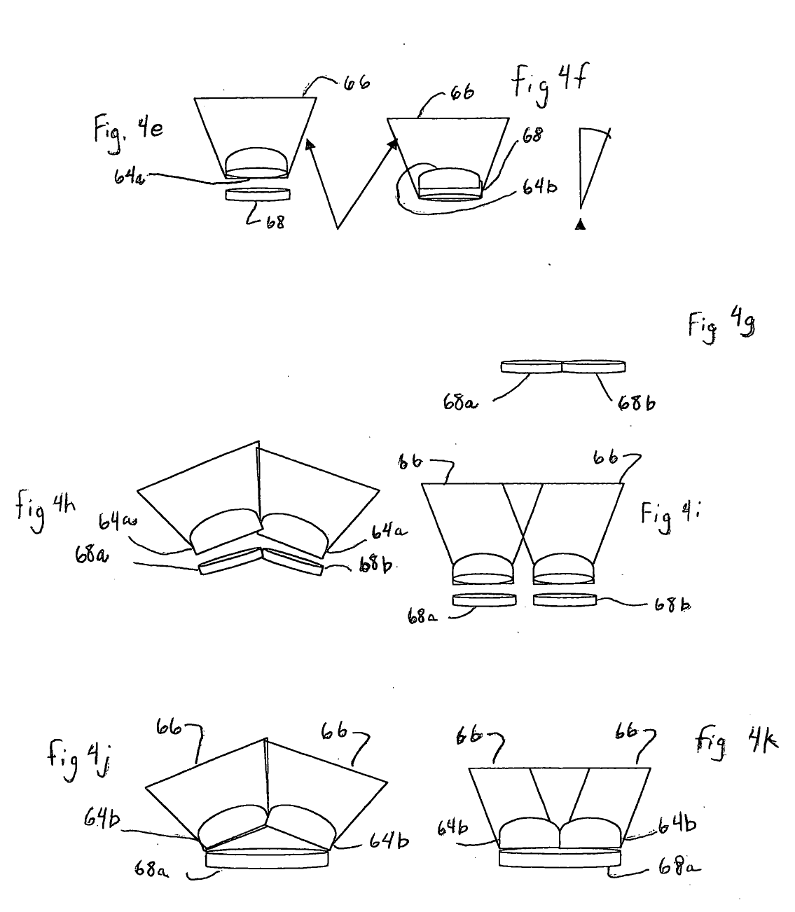

FIG. 4a depicts two linear image acquisition devices 6a and 6b, each of them with an input adjustment device 36b and 36b disposed at the end of the linear image acquisition device 6a and 6b. Each input adjustment device 36a and 36b provides a mechanism to manipulate the image it acquires through the image acquisition device 6a or 6b. The input adjustment device 36a and 36b may be either a mirror or a lens or a combination of one or more of both of them to amplify and/or focus the image. In one embodiment, lenses are provided which enable magnification of the image in a range from 0 to 40 times magnification. This magnification capability can be applied to all lenses or to a single lens. In the case of a binocular optical device which includes two image acquisition devices 6a and 6b or where only two of a plurality of image acquisition devices are selected for operation, the magnification may be applied to a single lens. This lens may provide magnification adjustable between zero and up to 40 times. This may be useful to provide multiple views of the same subject or collection of subjects. In another embodiment, the input adjustment devices 36a and 36b may be configured to provide digital manipulation of the image to amplify, magnify or focus as desired by the user. This digital manipulation may be provided in combination with, or as a substitute for, one or more mirrors and/or lenses for manipulation of an image.

FIG. 4b depicts various views that may be obtained where three image acquisition devices are provided. In FIG. 4b, the right view field 42a is depicted R, and the left view field 42b is depicted L, while the rear or posterior view field 42c is depicted P. A lens 44a, 44b, or 44c is associated with each of the respective view fields, 42a, 42b, or 42c. From FIG. 4b it can be appreciated how a user would be provided an advantage over systems which include only two view fields, by way of an additional viewing field with the posterior view 42c. One can also appreciate how the left view field 42b and the right view field 42a may combine to give a complete, or seamless view field. In one embodiment, the left view field 42b and the right view field 42a combine to give a field from approximately 0 to 180° while the posterior 42c view field may be used to give a view field from approximately 190 to 350°. Alternatively, the various fields 42a, 42b, and 42c may be configured so that one or more of them overlap, or so that each of them is entirely separate.

FIG. 4c provides another depiction of the various views 42a, 42b, and 42c that may be obtained in accordance with certain embodiments of the invention. As can be seen in FIG. 4c, the optical device (not shown) may be arranged so that the various views 42a, 42b and 42c are essentially contiguous with one another. Alternatively, two or more of the views 42a, 42b, and 42c may overlap, or the views 42a, 42b, and 42c may be entirely separate from one another.

FIG. 4d shows a lateral internal view of an optical device 50 in accordance with an embodiment of the invention. The optical device 50 is provided with at least one image transmission apparatus such as optic cable 52, which is preferably flexible. The optical cable 52 may optionally be provided with an image capture mechanism 54, such as a camera or lens. One or more light sources 56a and 56b may also be provided to illuminate the area under examination or surgery. Reflective surfaces, such as mirrors 58a and 58b, may also be provided as necessary or desirable. The mirrors 58a and 58b may be used to redirect the light from the light sources 56a and 56b or to redirect an image into the image transmission apparatus such as optic cable 52. A lens 60 may be provided to further modify and adjust one or both of the light from the light sources 56a and 56b or the image being directed to the image transmission apparatus such as optic cable 52. Further, a protective lens 62 may be provided on or near the outside of the optical device 50.

Thus, one can appreciate that multiple lenses may be used to increase the viewing field of the device. These multiple lenses may be or may include a spheroid lens cover or a spheroid lens. The spheroid lens cover 64a and spheroid lens 64b are shown in FIGS. 4e and 4f, respectively. Each of these may serve as a wider optical lens which will amplify and/or expand the viewing field 66 of the internal lens 68. In particular, the spheroid lens cover 64a and/or spheroid lens 64b may be used to expand the visual field 66, for instance, to 180°, effectively expanding the visual field 66 into a trapezoidal shape with a greater width at the edge further away from the internal lens 68. The internal lens 68 may be used to magnify or otherwise modulate the viewing field 66.

Further, the spheroid lens cover 64a and/or spheroid lens 64b may be stacked as shown in FIGS. 4g, 4h and 4i, along with stacked internal lenses 68a and 68b. As shown in FIG. 4h, the internal lenses 68a and 68b are positioned at an angle relative to each other and two spheroid lens covers 64a are provided. FIG. 4i depicts another arrangement, wherein the internal lenses 68a and 68b are positioned laterally so that the viewing fields 66 overlap.

FIGS. 4j and 4k depict spheroid lens 64b arrangements similar to those shown in FIG. 4i, with the exception that the embodiments shown in FIGS. 4j and 4k use a single internal lens 68a with multiple spheroid lenses 64b. As one skilled in the art would appreciate, a variety of arrangements are feasible. In FIG. 4j, the spheroid lenses 64b are stacked to arrive at generally adjacent viewing fields 66. FIG. 4k shows the spheroid lenses 64b stacked so that the result in overlapping viewing fields 66.

The stacking concept described above may be applied to any variety of optic faces as desirable or necessary. Additionally, the spheroid lenses and lens covers described above may be provided with further protective lens covers which are shaped so as to provide no optical modulation or distortion. Rather, these protective lens covers serve to protect the optics from their external environment.

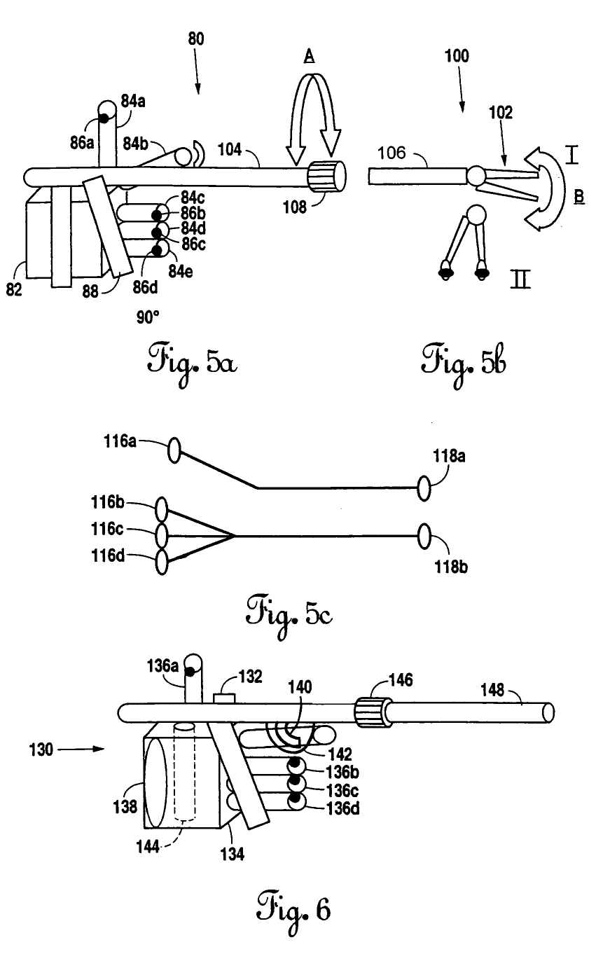

In another aspect of the invention, a medical device is provided which allows a surgeon to manipulate one or more tools inside a patient, while inserting little more than the tool portion of the device within the patient. FIG. 5a depicts a medical device 80 in accordance with such an embodiment of the invention; In this embodiment, a hand piece 82 is provided that accepts either a right hand, or a left hand of a user. In certain embodiments, the hand piece 82 is configured to accept a gloved hand. The hand piece 82 may also be configured bilaterally, to universally accept either hand of a user. In certain embodiments, the hand piece 82 may be sized so as to accept a particular hand size, for instance, a size 6-8 hand as measured for surgeon's gloves. The hand piece 82 may include separate portions for a user's fingers 84a-e. One or more of these portions for a user's fingers 84a-e may optionally be combined, so that a user's fingers rest within a mitten-like area (not shown).

In one embodiment, one or more pressure sensors 86a-d are provided. Each pressure sensor 86a-d detects the amount of pressure being applied by the relevant finger. This information is passed along to a mechanism (not shown) associated with the instrument portion (not shown) which operates a tool (not shown). The mechanism is responsive to the amount of pressure being applied by a particular finger and adjusts the operation of the tool accordingly. In this way, when a user exerts more force upon a pressure sensor 86a-d, a greater force is implemented by the tool. In such an embodiment, the tool may be such that one aspect of the tool is fixed. For instance, where the tool is forceps, one prong of the forceps may be fixed, and the other mobile. Alternatively, or in combination with this pressure system, the tool and mechanism may be configured to transmit to the user any resistance to the pressure being applied by that user. For instance, if the tool is a forceps, once the forceps reach a point at which they can no longer close any further due to the complete compression of the tissue or other material being grasped, the lever 88 being used to operate the forceps would similarly not close any further. In this way, the user experience is as though the user were working directly with the material being manipulated, rather than through the various mechanical or electronic linkages provided by the medical device 80.

The medical device includes an instrument portion 100 (shown in FIG. 5b), and one or more devices or control elements to allow a user to manipulate a tool (not shown). The tool is associated with the instrument portion 100 or some other feature of the medical device 80. In one embodiment, a trigger (not shown) and a lever 88 are provided to manipulate the tool. In an embodiment with a trigger, the trigger may be manipulated by squeezing a finger of a user by to cause some operation of the tool. For instance, squeezing the trigger may cause a tool to rotate. The lever 88 may be operated by squeezing one or more fingers, thereby also manipulating the tool. In alternate embodiments, the operations of the lever 88 and the trigger may be reversed or may be otherwise provided by buttons or switches or some other control element or combination of control elements.

In FIG. 5b, the instrument portion 100 is shown. The instrument portion 100 houses and includes mechanisms for manipulation and operation of a tool 102. A trigger (not shown) may operate to cause the tool 102 to rotate in the direction of arrow B, from position I to position II. In one embodiment, the tool 102 rotates to one of several preset positions, for instance, the tool 102 may be at an angle of 0, 45, or 90 degrees in relation to the instrument portion 100. When set at an angle of 0 degrees, the tool 102 is in a straight line in relation to the instrument portion 100. In another embodiment, the tool 102 may be set at anywhere between an angle of from 0 degrees to about 90 degrees in relation to the instrument portion 100.

Referring to FIGS. 5a and 5b, the interaction of the various components of the device may be more fully appreciated. A transcutaneous shaft 104 engages an internal shaft 106. The internal shaft 106 is optionally removably attached to the transcutaneous shaft 104 and linearly extends away from the transcutaneous shaft 104. The internal shaft 106 is attached to the tool 102. The tool 102 may optionally be detachable from the internal shaft 106. A knob 108 may be provided and is operable to cause the internal shaft 106, and any attached tool 102, to rotate, for instance in the direction of arrow A. Alternatively, the knob 108 may be provided further up the transcutaneous shaft 104, or the function of the knob 108 may be provided by some other control element provided with the hand piece 82.

In the embodiment of FIG. 5b, the tool 102 is depicted as forceps. In accordance with the present invention, the tool 102 may be a wide variety of other useful devices, especially where such devices are useful in medical procedures. For instance the tool 102 may be forceps, flat scissors, curved scissors, right angle scissors, DeBakey-type forceps, right angle forceps, blunt forceps, curved clamps, angular clamps, an ultrasound probe, a laser, a cautery device, a staplers, a knife, a suturing device, a rivet driver, a ligation device, an aspiration device, an injection device, a biopsy device, a radiotherapy device; or a radioactive emitter loading device. The tool is preferably one of three types. The first type of tool is configured for a single use on a patient and is then discarded. A second tool type is replaceable and preferably sterilizable. In such a device the entire instrument may be cleaned and sterilized for repeated use or the tool itself may be removed for cleaning and sterilization. In a third tool type, a variety of different tools may be interchangeably used with a single device, and may be detached and reattached to the device as required by a user.

FIG. 5c depicts an schematic showing an arrangement of pressure sensors that may be used in accordance with certain embodiments of the invention. In the arrangement of FIG. 5c, pressure sensors 116a-d are provided for the thumb and first three fingers, respectively. As described above, the mechanism which operates the tool is responsive to the amount of pressure being applied by a particular finger and adjusts the operation of the tool accordingly. The mechanism which operates the tool may be either mechanical or electrical in nature, or may be a combination of both.

Pressure sensors 118a and 118b may also be provided for the tool of a medical device, in accordance with certain embodiments of the invention. In one embodiment, the tool is a pair of forceps, one of which is fixed and the other mobile. The pressure sensors 118a and 118b may be used in such an arrangement to detect the pressure being placed upon the forceps. The pressure sensors 118a and 188b are configured so that the pressure they sense is transmitted back to the user, through a mechanical or electrical mechanism, as previously described. Alternatively, the mechanism used to transmit the pressure signal generated by the pressure sensors 118a and 118b is both mechanical and electrical in nature.

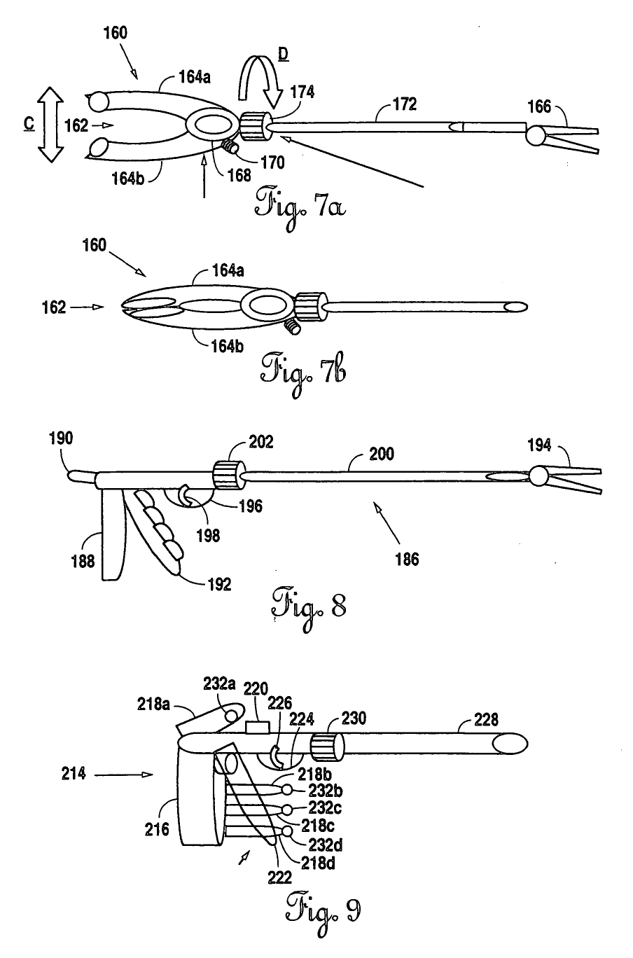

Referring to FIG. 6, another embodiment of a medical device 130 is depicted. This device 130 includes a cauterization button 132. The cauterization button 132 is associated with a mechanism in the tool (not shown) which causes at least a portion of the tool to heat up so as to permit a user to cauterize tissue. In one embodiment activation of the cauterization button 132 closes an electrical circuit (not shown) so that a voltage is delivered to the tool which is then ready to provide electro-cautery to tissue. Once the cauterization button 132 is deactivated, the electricity is no longer delivered to the tool and the tool cools off. The medical device 130 is generally insulated from the electric circuit so that an electrical charge is not unintentionally transmitted to other parts of the medical device 130 or to the user. Optionally, the function of the cauterization button 132 may be provided by a different control element or feature.

Where the hand piece 134 includes separate finger portions 136a-d, the hand piece 134 may be configured so that a user's hand fits entirely or partially with the hand piece 134. In such an embodiment, the hand piece 134 includes an interior portion (not shown) configured to accept a user's hand, and preferably to accept a gloved hand. The interior portion is accessed through an opening 138 in the hand piece 134 of sufficient size to allow a user's hand, or part of a hand, to enter the interior portion of the hand piece 134. The trigger 140 and trigger guard 142 are positioned for easy access by the user's index finger. A grip, or palm rest 144 may also be provided within the interior space of the hand piece 134. The grip or palm rest 144 facilitates easy manipulation of the medical device 130, and helps to provide the user a firm grasp of the device 130, and prevent slippage. A rotation knob 146 for the internal shaft 148 is also depicted in this view.

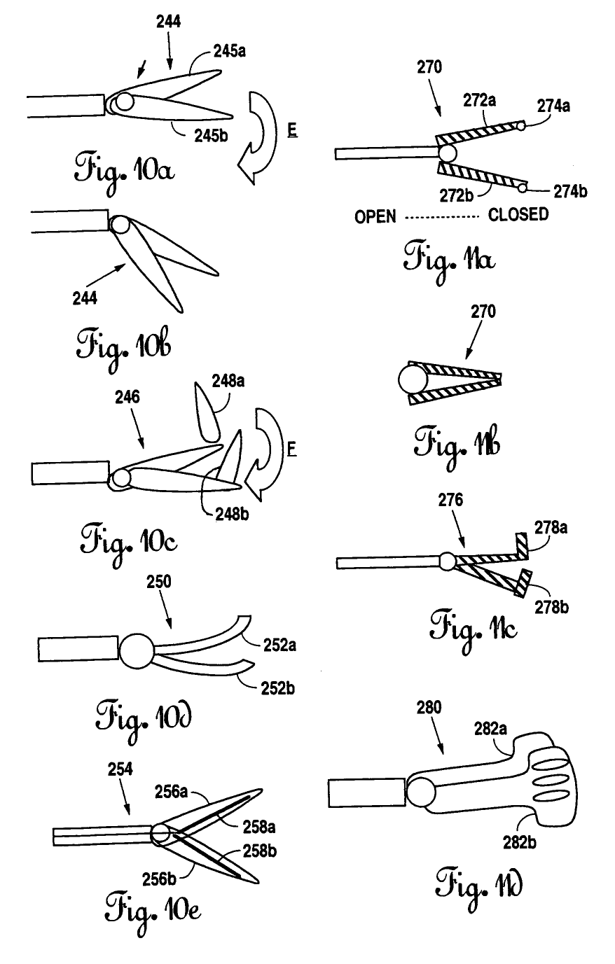

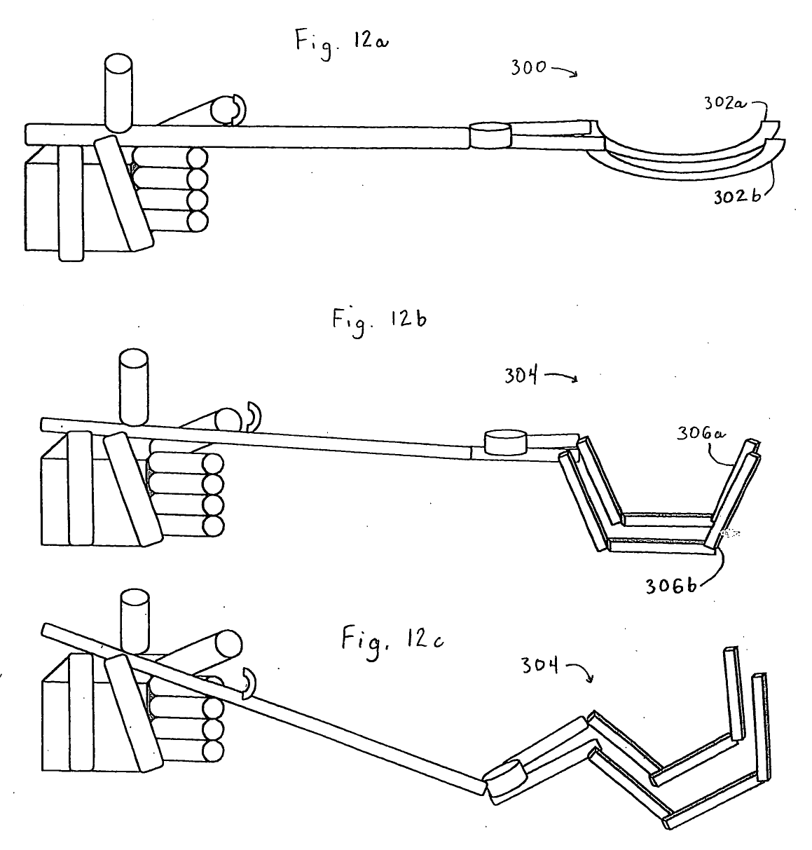

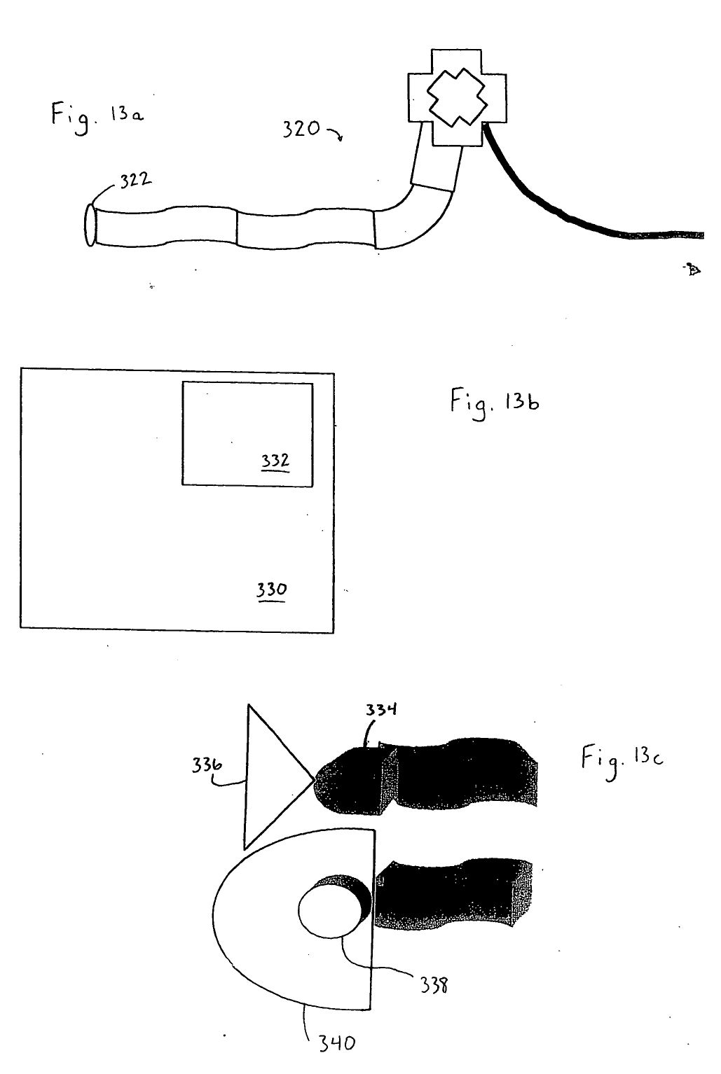

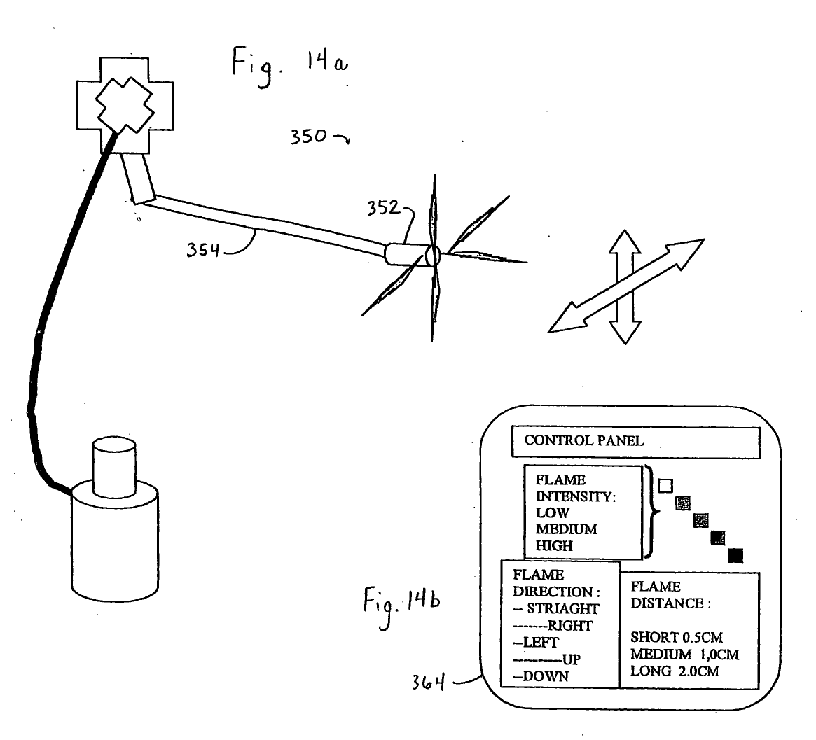

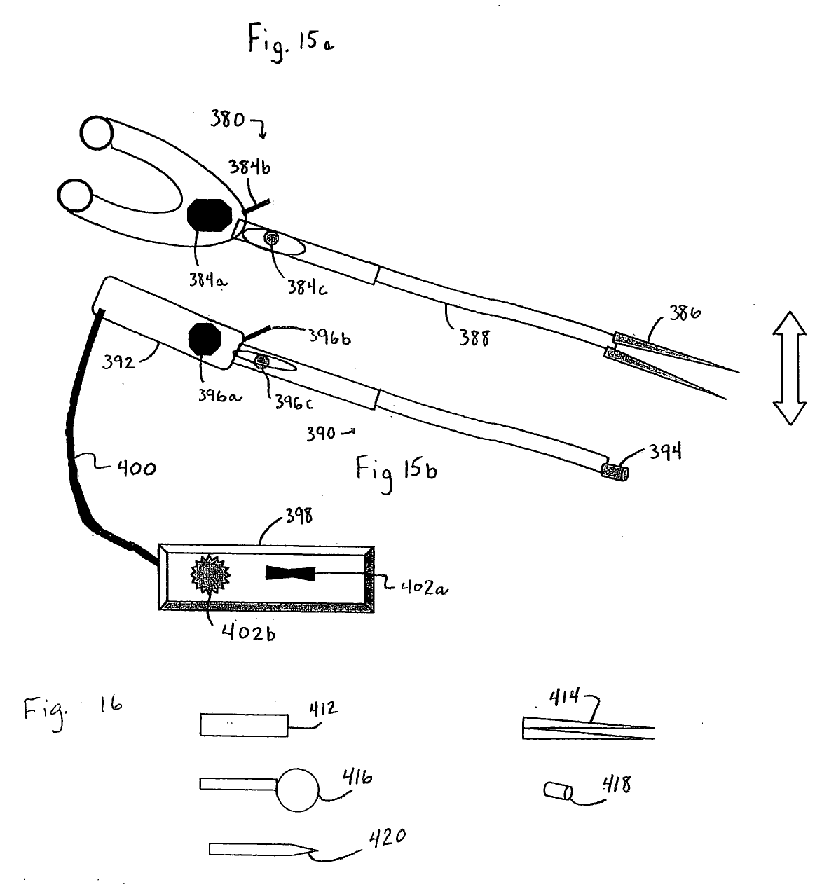

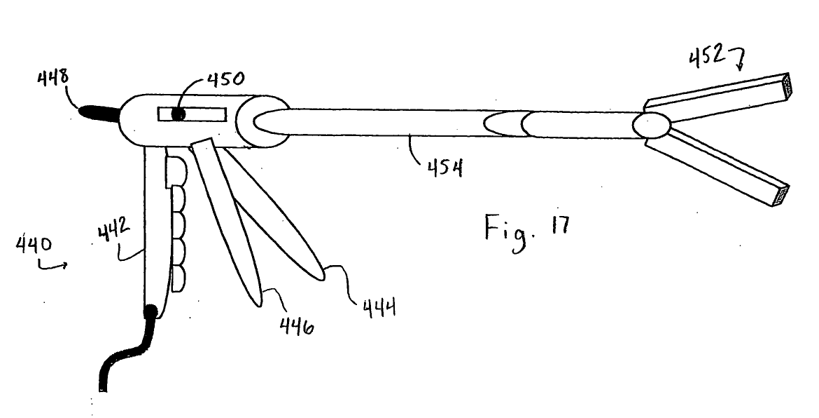

FIG. 7a depicts a medical device 160 in accordance with another embodiment of the invention. A scissor-type hand piece 162 is provided with a first elongated portion 164a and a second elongated portion 164b. Together, the first elongated portion 164a and the second elongated portion 164b are moveable towards each other, much like the operation of the handle of a pair of scissors. Movement of these elongated portions 164a and 164b in the path of arrow C operates the tool 166. A cauterization button 168 is provided and operates in a manner similar to that previously described. Another button 170 rotates the tool 166 at an angle relative to the device 160. This button 170 operates in a manner similar to that previously described with respect to the trigger of other embodiments of the invention. This embodiment is depicted with an alternative single shaft 172, which is rotatable in relation to the hand piece 162, for instance in the path of arrow D. A rotation knob 174 is provided to facilitate a user's rotating the shaft 172. As with the other embodiments, the control elements are not limited to the particular arrangement shown.

FIG. 7b shows a lateral view of the medical device 160 of FIG. 7a in a different position. In FIG. 7b, the scissor-type hand piece 162 is in a closed position. In particular, the first elongated portion 164a and the second elongated portion 164b are moved toward each other.

FIG. 8 depicts a pistol-handled medical device 186 in accordance with another embodiment of the invention. In this device 186 a pistol style handle 188 is provided which fits into the palm of a user's hand. In other respects, the medical device 186 is similar to those previously depicted. A cauterization button 190 is provided, as is a lever 192 for operation of the tool 194. A trigger guard 196 is also provided, as is a trigger 198 for rotation of the tool 194 in relation to the device 186. This embodiment is depicted with a single transcutaneous shaft 200 and a detachable tool 194 affixed to the end of the single shaft 200. A rotation knob 202 is also provided to facilitate rotation of the single shaft 200 and the tool 194 thereon.

FIG. 9 depicts another medical device 214 with a palm/wrist circumferential band 216 and sleds 218a-d for the thumb and middle, ring, and little fingers. The band 216 is useful to secure the device 214 to a user's hand and to prevent slippage. In other respects, the medical device 214 is similar to that depicted in FIG. 8. A handle (not shown) may be provided for a user to grip with the hand much like the pistol style handle of the embodiment shown in FIG. 8. A cauterization button 220 is provided, as is a lever 222 for operation of the tool (not shown). A trigger guard 224 is also provided, as is a trigger 226 for rotation of the tool in relation to the device 214. This embodiment is depicted with a single transcutaneous shaft 228, and a rotation knob 230 is also provided to facilitate rotation of the shaft 228 and the tool thereon.

The sleds 218a-d may be provided with pressure sensors 232a-d in the medical device 214 of FIG. 9. These pressure sensors 232a-d operate in substantially the same manner as that previously described in FIGS. 5a and 5c, providing sensory input between the user and the device with regard to the tissue density being encountered by the tool (e.g., soft tissue, firm tissue or bone) or the pressure being applied by the user upon the device 214. The sleds may be constructed so as to achieve functionality similar to that achieved with the glove-handle of FIG. 5a.

Referring to FIGS. 10a, 10b, 10c, 10d and 10e a variety of scissors-type tools for use with certain embodiments of the present invention are provided. FIG. 10a shows a flat scissors tool 244 with a first cutting element 245a and a second cutting element 245b. This scissors tool 244 may be provided in a variety of sizes, for instance as a small version of Mayo-type scissors. The scissors tool 244 may be rotated, for instance in the direction of arrow E. After such a rotation the scissors tool 244 may be in the position shown in FIG. 10b. FIG. 10c shows a right-angled scissors tool 246 with a first cutting element 248a and a second cutting element 248b. This scissors tool 246 may be provided in a variety of sizes and may function similar to Potts-style scissors. Further, this scissors tool 246 may be rotated, for instance in the direction of arrow F. FIG. 10d shows a curved scissors tool 250 with a first cutting element 252a and a second cutting element 252b. Each of the first cutting element 252a and second cutting element 252b are provided with a curved shape. This scissors tool 250 may also be provided in a variety of sizes and may function similar to dissection scissors, such as Metzenbaum-type scissors. FIG. 10e shows a cauterizing scissors tool 254 with a first cutting element 256a and a second cutting element 256b. Additionally, each cutting element 256a and 256b includes a heating element 258a and 258b. Any of the foregoing scissors tools may be configured with heating elements for cauterizing tissue. Further, many of the other tools contemplated for use with the various medical devices of the invention may be provided with heating elements for cauterizing tissue. Each of the scissors tools 244, 246, 250, and 254 may be rotated. This rotation may include both rotation about the axis of the shaft to which the tools are mounted and about an axis at an angle to that shaft, as described with respect to the previous tool embodiments.

Referring to FIGS. 11a, 11b, and 11c, a variety of forceps tools for use with certain embodiments of the present invention are provided. FIG. 11a shows a DeBakey style forceps tool 270 with a first grasping arm 472a and a second grasping arm 472b. This forceps tool 270 may be configured so that it is suitable for vascular work, and other fine tissue handling. For delicate work, the forceps tool 270 may be configured to deliver only limited amounts of force, and need not be configured to deliver a crushing level of force. In one embodiment, the forceps tool 270 is provided with pressure sensors 274a and 274b which are useful to transmit a pressure sensation from the forceps tool 270 back to the user, as previously described, especially with reference to FIGS. 5a and 5c. FIG. 11b shows the forceps tool 270 of FIG. 11a in a closed position.

FIG. 11c depicts a right-angled forceps tool 276 with a first grasping arm 278a and a second grasping arm 278b. Preferably this forceps tool 276 is constructed from a material with characteristics that are or are similar to metal or metallic materials, especially with regard to the material's density, strength, and flexibility. The tips of the forceps tool 276 may be configured in a variety of shapes, including fine, sharp tips, larger smooth blunt tips, or large blunt curved or right-angled tips.

FIG. 11d shows a blunt forceps tool 280 with a first grasping arm 282a and a second grasping arm 282b. This forceps tool 280 may be configured similarly to blunt tip clamps often used in open procedures, such as a Babcock-style clamp. Each of the forceps tools 270, 276, and 280 may be rotated as described above, both about the axis of the shaft to which they are mounted and about an axis at an angle to that shaft, as described with respect to the previous tool embodiments.

Preferably, the forceps tools 270, 276, and 280 may be configured to mimic the action, and provide the sensation and operation of a common “open handed” forceps tool. The forceps tools 270, 276, and 280 may be provided with spring action which must be overcome in order to close or approximate the forceps tool 270, 276, and 280. This spring action may provide a user with feedback useful to determine the mechanical pressure needed to close the forceps tool 270, 276, and 280. The forceps tools 270, 276, and 280 may otherwise be provided with pressure sensors such as those depicted in FIG. 11a as part of an electrical or mechanical pressure sensor-based feedback mechanism, as described previously. Further, the forceps tools 270, 276, and 280 may include a combination of pressure sensors based feedback mechanisms and purely mechanical feedback mechanisms. An example of such a purely mechanical feedback mechanism would be the resultant feedback provided by a mechanical linkage as the tool reaches the limit of its range of motion. Where the control is mechanically linked to the tool, movement of the control is limited as the movement of the tool is limited.

Referring to FIGS. 12a and 12b, clamp tools for use with certain embodiments of the present invention are provided. FIG. 12a shows a Cooley style clamp tool 300 with a first clamping arm 302a and a second clamping arm 302b. FIG. 12b shows a Satinski style clamp tool 304 with a first clamping arm 306a and a second clamping arm 306b. This clamp tool 304 includes at least one angle in the clamping arms 306a and 306b, which in some embodiments is from about 75 to 85 degrees. Each of the clamp tools 300 and 304 may be rotated as described above, both about the axis of the shaft to which they are mounted and about an axis at an angle to that shaft, as described with respect to the previous tool embodiments. FIG. 12c depicts clamp tool 304 of FIG. 12b, in a different position, after the clamp tool was rotated.

While the clamp tools 300 and 304 may be configured similarly to the style of clamps used in open surgery, they are specially adapted for minimally invasive surgery. In certain embodiments the clamp tools 300 and 304 are adapted to collapse into a small size so as to fit through a trocar port, or other surgical incision, and then to expand upon deployment within a patient's body. A mechanical mechanism or combination of mechanisms may be provided to close or approximate the tips of the clamp tools 300 and 304. The mechanical mechanism or mechanisms transmit the force of pressure applied by the user and preferably are adapted to provide the user feedback on the amount of resistance delivered to the clamp tools 300 and 304. The clamp tools 300 and 304 may be configured so that they may be locked at a particular degree of closure or approximation.

Referring to FIG. 13a, an ultrasonic medical device 320 is provided. The ultrasonic medical device 320 is useful to image various tissues and structures of a patient. The device 320 can image solid, hollow, or blood or fluid filled structures. The device 320 can also measure the flow rate of vascular structures and can be used to provide graphic diagrams of tissue based on the selection of a user. For instance, a user may elect to view hepatic, lung, bone, bowel, spleen, vessel, ovary, uterine, or a variety of other types of tissue. The device 320 may be configured with a probe tip 322 that is smaller than that currently available for use with ultrasonic devices. Further, the device 320 is adapted for manipulation with a single hand so that it is easy to position and use.

The ultrasonic medical device 320 may be used in combination with an optical medical device, or other minimally invasive medical device as described herein. When use with an optical medical device the images produced by the devices may be combined in a single display for the convenience of the user. FIG. 13b depicts a screen image with a combination of an visual image 330, such as that obtained by a camera, and an inset ultrasonic image 332. The ultrasonic image 332 may replace the visual image 330 entirely and the ultrasonic image 332 may include a diagrammatic depiction of the subject being imaged. For instance, the ultrasonic image 332 may include an outline of vessels with the pulse and/or flow rate determined and displayed along with the ultrasonic image 332. The volume or size of fluid filled and other structures may also be calculated and depicted along with the ultrasonic image 332.

FIG. 13c depicts two ultrasonic probe types for use in accordance with certain embodiments of the invention. One is a curvilinear probe 334 which images a cone shaped area 336 that progressively increases with increasing distance from probe 470. A second type is a spherical probe 338 which images a rounded area 340 that generally extends in a 180 degree arc away from the spherical probe 338. A third type of probe suitable for use with certain embodiments of the invention is a flat probe (not shown) which is useful for imaging vascular structures. In one embodiment, an ultrasonic device is provided that has may be used with multiple detachable probes, such that any of a variety of ultrasonic probes may be used.

FIG. 14a depicts a laser 350 in accordance with another embodiment of the invention. The laser 350 may be any suitable type of laser, and in one embodiment is an argon laser. The laser 350 is specially adapted for minimally invasive surgery to apply laser energy to a target selected by the user. The laser 350 is equipped with a tip 352 that is mobile, so that the user may maneuver it. In this way, the laser 350 may be positioned for delivery of laser energy in the vision field provided by a second medical device. The tip 352 may be attached to a flexible wand 354 which further facilitates convenient manipulation and direction of the laser energy. The laser 350 may be multi-directional such that laser energy is directed into one of several discreet areas surrounding the tip 352.

FIG. 14b depicts a display screen 364 showing a control panel useful for controlling a laser, such as that depicted in FIG. 14a. The control panel is associated with a controlling computer (not shown) and allows a user to select from various laser intensity levels and effective distances from the laser tip. The control panel also provides a user the ability to select the direction of emission of the laser energy in relation to the laser tip. The control panel may be touch sensitive, or it may be provided with a separate keyboard and/or pointing device, such as a mouse (not shown) for the user to input instructions to the controlling computer.

In another embodiment, the present invention provides tools for tissue cautery. FIG. 15a depicts a view of one embodiment of a forceps-type cautery device 380. A scissors-style handle is provided with a first elongated portion 382a and a second elongated portion 382b similar to that shown in FIGS. 7a and 7b. A variety of controlling elements 384a-c such as knobs, buttons, or switches are provided to control the tool 386. A first controlling element 384a may be used to activate or deactivate cautery. A second control element 384b may be used to orient the tool 386, for instance rotating the tool 386 at an angle relative to the shaft 388 to which the tool 386 is mounted. A third control element 384c may be used to cause the tool 386 to retract or advance relative to the cautery device 380. The tool 386 shown in the embodiment in FIG. 15a is a forceps tool 386, however a variety of other tools might also be used.

FIG. 15b depicts another cautery device 390. This cautery device 390 is similar to that depicted in FIG. 15a, however a wand-type handle 392 is provided. This handle 392 allows the device 390 to be used by either hand singularly. The tool 394 in this embodiment is a cylindrical cautery tool. A variety of controlling elements 396a-c may also be provided. A separate cautery controller 398 may also be provided and is connected to the cautery device 390 by a cord 400. The cautery controller 398 may include a variety of controller elements 402a and 402b which allow a user to adjust the intensity of the energy delivered to cauterize tissue or to turn the cautery function on or off, or to control some other aspect of the cautery device 390. Although only described with reference to cautery device 390, the cautery controller 398 may be used with a wide variety of cautery devices.

FIG. 16 shows a variety of different cautery tool tip types. These cautery tool tip types include the following: a flat tool 412 which is a dull square blade; a forceps tool 414 similar to the forceps cautery tool used in open surgery; a spherical ball tool 416 which provides a greater surface area for cauterizing more tissue; a rounded tool 418 which is blunt and may be provided as a relatively small tool; and a needle tool 420 for cutting or performing fine dissections. These cautery tools are configured so that they be used for probing, touching, and moving tissue without damage until the cautery function is activated by the user.

FIG. 17 depicts a stapler 440 in accordance with an embodiment of the invention. The stapler 440 is constructed to staple tissue and/or cut between the staple lines. The stapler 440 allows a user to simply staple tissue, or staple and then cut the stapled tissue. Alternatively, a user may decide to staple tissue and then cut the tissue using a cautery scissor or cautery wand to divide the tissue between the staple lines. The stapler 440 provides superior staple lines and is appropriately sized to be of particular use in minimally invasive surgical techniques. The stapler 440 includes a knife (not shown) that may cut in either a cold (ambient temperature) setting or a hot (e.g., a cautery temperature) setting. The stapler 440 may be configured for single handed operation.

In the stapler 440, a handle 442 is provided that, in one embodiment, may be gripped by either the right or left hand of the user. A double lever system with a close lever 444 and a staple lever 446 is also provided. The close lever 444 operates to close the stapler about the tissue to be stapled. The staple lever 446 operates to actually staple the tissue together. A cut controlling element, for instance a button or switch 448, engages the cutting function of the stapler 440 and a position controlling element 450 allows a user to rotate the position of the stapler tool 452 in relation to the stapler shaft 454. The staple function and the cutting function of the stapler 440 may be manually or automatically driven. When the staple function is automatic, it may be either gas or electric or any other suitable method of automatically driving staples.

In one embodiment, the stapler tool 452 may be positioned at an adjustable angle from 0 to 90 degrees in relation to the stapler shaft 454. In an alternate embodiment, the stapler tool 452 may be positioned at a preset angle of either 0, 45, or 90 degrees in relation to the stapler shaft 454.

The stapler tool 452 may also include a lock function which operates to ensure that the tissue to be stapled and/or cut is not squeezed out before being stapled. If the tissue is squeezed out as the stapler is closed, a complication may arise resulting in bleeding or leakage from the staple line. FIG. 18a depicts an embodiment where a lock 460 functions to substantially hold and secure the tissue in place before stapling and/or cutting. In FIG. 18a, the lock 460 is shown in an open position. FIG. 18b depicts the same embodiment as FIG. 18a, with the lock 460 in a closed position. In the closed position, the lock 460 is dropped on the two anvils 462a and 462b to hold the tissue in place. The anvils 462a and 462b are mounted in a V-configuration in relation to each other and one end of each of anvils 462a and 462b moves toward the other in order to staple tissue together. The use of the lock significantly increases the integrity and security of the staple line.

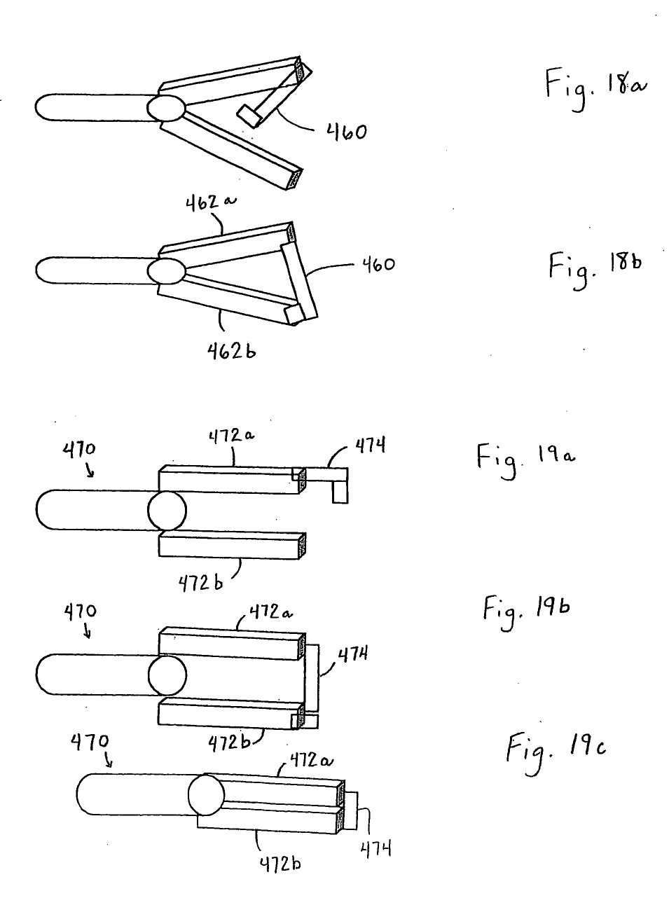

FIGS. 19a, 19b and 19c depict an alternate embodiment of a portion of a stapler device 470. In this embodiment, the anvils 472a and 472b are mounted in a parallel arrangement to each other and the entire anvil 472a or 472b moves toward the other anvil 472a or 472b in order to staple tissue together. FIG. 19a depicts a stapler 470 with the anvils 472a and 472b in an open position. The lock mechanism 474 is also in an open position. The lock mechanism of FIGS. 19a, 19b, and 19c operates similarly to that of FIGS. 18a and 18b, wherein the lock mechanism prevents tissue from being squeezed out of position when the anvils 472a and 472b are closed, but before the tissue is stapled. FIG. 19b depicts the anvils 472a and 472b in an open position, with the lock mechanism 474 closed. In the position shown in FIG. 19b, the tissue is held in position by the lock mechanism 474, however the anvils 472a and 472b are not yet closed together to effect driving a staple through the tissue. FIG. 19c depicts an embodiment of a stapler 470 with parallel anvils 472a and 472b in a closed position. In FIG. 19c, anvils 472a and 472b are positioned almost immediately next to one another.

Optionally, the stapler 470 may staple and cut the tissue. In one embodiment the cut is performed with an unheated razor blade (not shown). Alternatively, the cut may be performed with a heated razor blade so that the tissue is cauterized as it is cut. Simultaneously cauterizing and cutting the tissue discourages excess bleeding from the cut tissue. Electrical energy may be applied to the blade or a separate cautery element (not shown) in order to sufficiently heat the tissue for cauterization.

FIGS. 20a and 20b depict arrangements for stapling tissue in accordance with certain embodiments of the invention. A double row of staple lines 490a and 490b is applied to one side of the cut 492 while a second double row of staple lines 494a and 494b is applied to the other side of the cut 492. The cut 492 is separate from the edges of the nearer staple lines 490b and 494b.

FIG. 20b depicts an additional option, where the tissue is compressed along a tissue compression lines 496a and 496b on the outermost side of the staple lines 490a and 494a. With this option, the tissue is compressed prior to stapling or cutting so as to reduce blood flow to the region before stapling or cutting. This has the added effect of increasing the success of the stapling to control and restrict blood flow in all vessels (arteries, veins, and capillaries). Further, the compression may help to keep the tissue immobile for the stapling procedure, thereby assuring the stapling procedure creates a strong and secure fastener for the tissue or tissues being joined or closed.

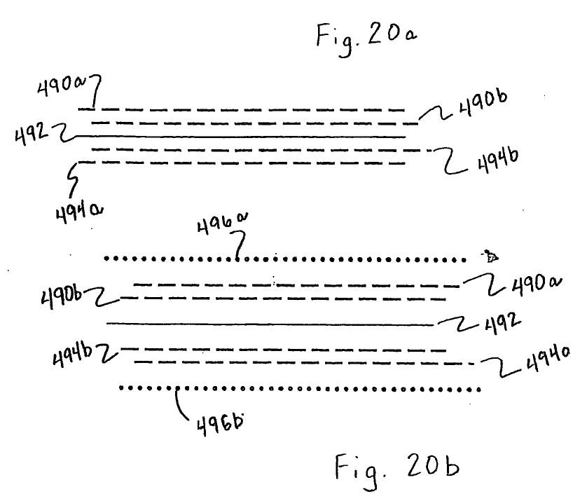

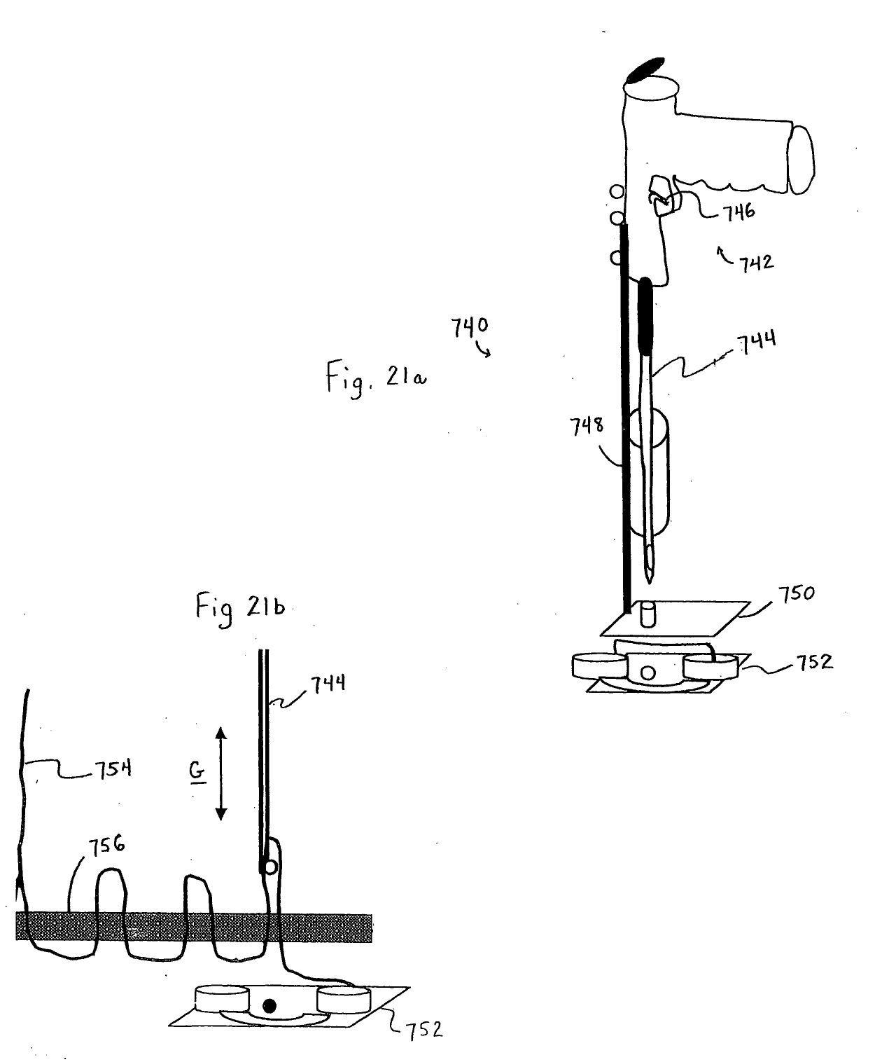

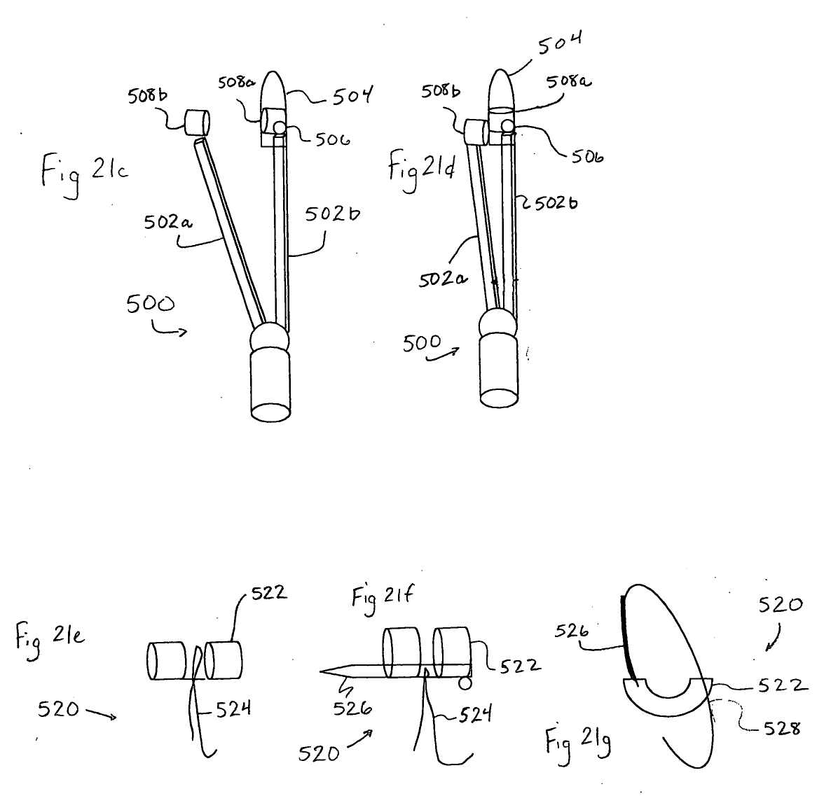

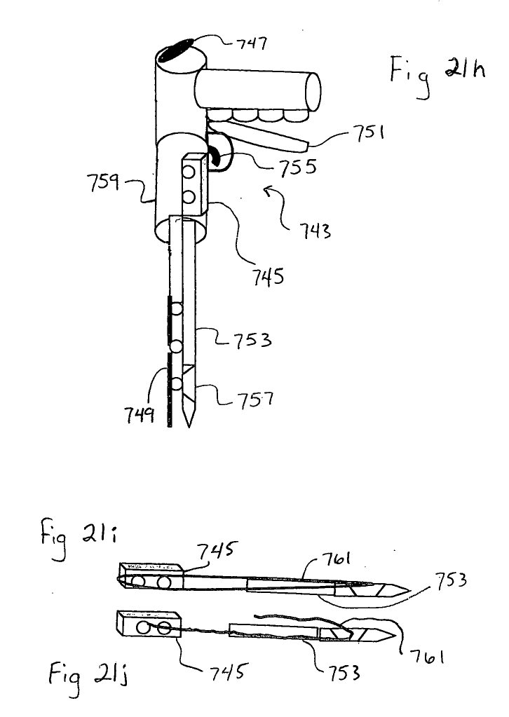

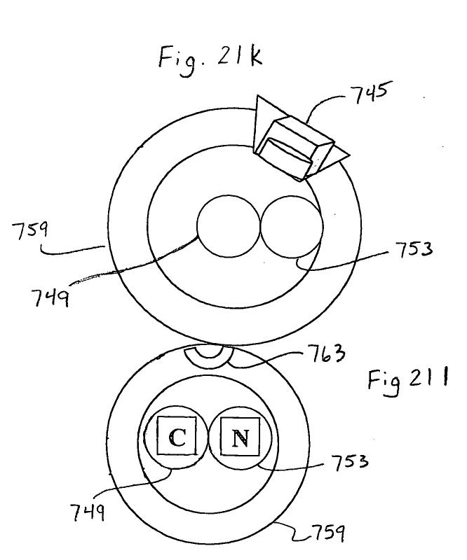

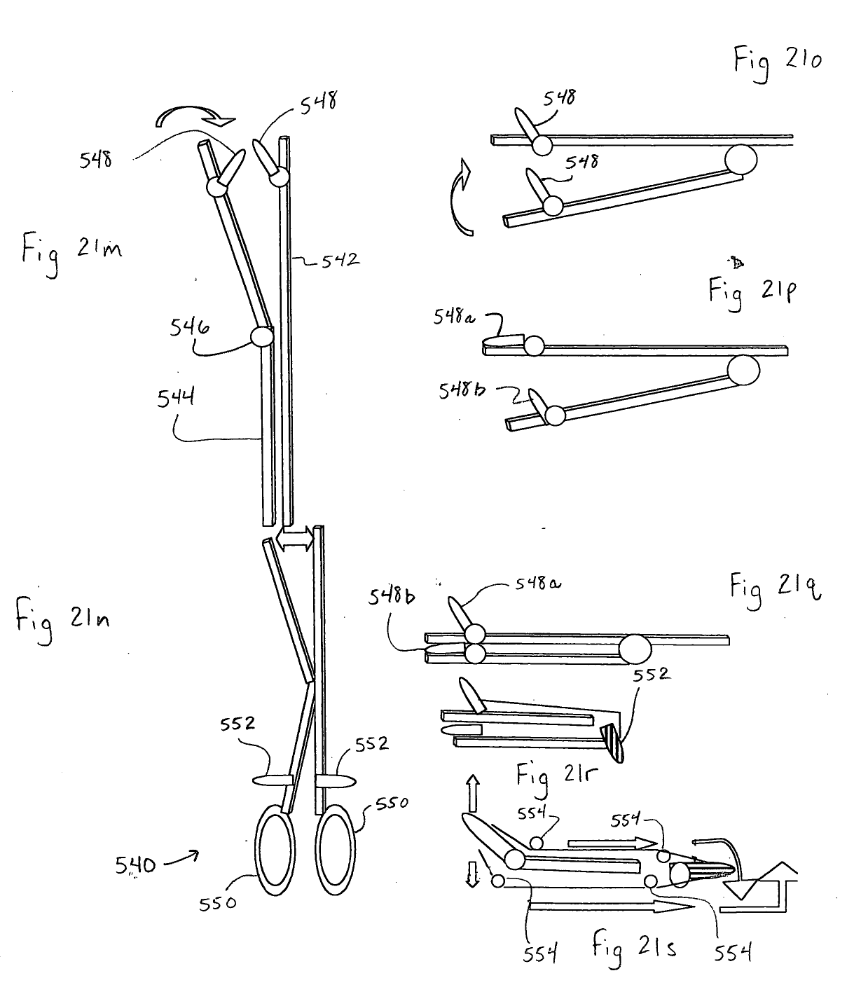

FIG. 21a depicts a suturing device 740 in accordance with an embodiment of the invention. A firing gun 742 is provided with a needle 744 which is adapted to push suture material through tissue and withdraw it. The firing gun 742 includes a trigger 746 which, with each pull, causes the firing gun 742 to push the needle 744 and attached suture material through the tissue and then withdraw it. Alternatively, each pull of the trigger 746 may cause only a portion of the needle's movement, for instance the pushing of the needle 744 through the tissue. Then a separate pull of the trigger 746 would cause the needle 744 to be withdrawn from the tissue. A shaft 748 may be used to secure the firing gun 742 to a base 750 with a bobbit 752. An internal bobbit 752 is depicted, although an external bobbit (not shown) may also be used.

FIG. 21b depicts a portion of the suturing device of FIG. 21a as it operates. In operation the suture material 754 is grasped by the needle 744 upon passage to the bobbit assembly 752 and is then pulled through the tissue 756. The needle 744 moves in the direction of arrow G. The needle 744 advances and after advancement, comes to a stop. With another trigger pull, the needle 744 withdraws. A portion of the suture material 754 is then on the other side of the tissue 756 (opposite the bobbit assembly 752) and is left in this position by the needle 744. The needle 744 is then moved laterally along the tissue 756 and may descend through the tissue 756 to retrieve another portion of suture material 754.

FIGS. 21c and 21d depict a needle driver device 500. The needle driver device 500 is constructed to accept a straight needle and/or a curved needle. It includes two driver arms 502a and 502b which are pivotally attached, for instance by a hinge. A retractable guard 504 may also be provided. A separate hinge 506 may also be provided for one or more of the needle holders 508a and 508b.

FIGS. 21e, 21f and 21g show a garrot style needle holder 520. FIG. 21e shows the garrot style needle holder 520 empty. A holder base 522 is provided, as is a garrot 524. In FIG. 21e, the garrot is in a loose position, while in FIG. 21f, the garrot 524 is tight around the needle 526. FIG. 21g shows an end view of the garrot style needle holder 520. The garrot in this embodiment is depicted as two components, a noose spring 526 and a noose 528. Preferably the noose 528 is constructed from a generally flexible material such as wire or nylon.

FIGS. 21m-21s depict yet another variety of a needle driver: an anvil-style needle holder 540. In FIG. 21m, a portion of an anvil-style needle holder 540 is shown. In this embodiment, a first jaw 542 is shown as an elongated jaw. A second jaw 544 is shown with a pivotal hinge 546 provided in the middle. The hinge 546 permits a portion of the second jaw 544 to move relative to the first jaw 542. Each of the jaws 542 and 544 is provided with a pivotally attached anvil 548. FIG. 21n shows a complete anvil-style needle holder 540. The two jaws 542 and 544 of FIG. 21m are shown, along with two rings 550 for grasping and manipulating the device. An anvil switch 552 is provided for each anvil 548, and is used to actuate the respective anvil 548. FIG. 21o shows a side view of a portion of an anvil-style needle holder with the jaws apart and the anvils 548 open. FIG. 21p shows a side view of a portion of an anvil-style needle holder with the jaws apart and one anvil 548a closed and one anvil open 548b. FIG. 21q shows a side view of a portion of an anvil-style needle holder with the jaws closed and one anvil 548b closed and one anvil 548a open. FIG. 21r shows a side view of a portion of an anvil-style needle holder with an anvil switch 552. FIG. 21s shows a system of pulleys and hin

A variety of needle sizes are suitable for use with certain embodiments of the present invention including: 6-0, 5-0, 4-0, 3-0, 2-0, 0-0, 1, and 2. Similarly, the needle 744 may be any of a variety of suitable types, including blunt, sharp, standard bevel, or inverted bevel. Suitable suture materials include dexon, polyglactic 910 (sold by Ethicon, Inc. under the tradename VICRYL), polydioxanone (sold by Ethicon, Inc. under the tradenames PDS and PDS II), nylon, stainless steel, or a monofilament material such as that sold by Ethicon, Inc., under the tradename Proline.

FIG. 21c depicts another arrangement for a suturing device 743. In this generally gun-shaped suturing device 743, a bobbit box cartridge 745 may be placed or loaded into the suturing device 743. Preferably the bobbit box cartridge 745 is constructed to snap into place. Various control elements are provided on the suturing device 743. In the particular arrangement shown, a button 747 is provided to cause a suture catcher 749 to advance. A handle lever 751 is provided to cause the needle 753 to advance. A trigger 755 is used to actuate the closing or opening of a bevel 757 on the needle 753. Preferably the needle 753 and suture catcher rod 749 are provided through the barrel 759 of the suturing device 743. This snap-in construction for the bobbit box cartridge 745 facilitates changing suture material and reuse of the suturing device 743 in a patient for multiple suture materials, for instance in different tissues.

FIGS. 21d and 21e depict arrangements for the bobbit box cartridge 745. In the arrangement shown in FIG. 21d, the bobbit suture 761 is a double strand which works in conjunction with the needle 753. The arrangement shown in FIG. 21e shows the bobbit suture 761 as a single strand which again, works in conjunction with the needle 753. Generally, the bobbit is optional and is preferably constructed to conserve space and minimize entanglement.

FIGS. 21f and 21g show cross-sectional views of the barrel 759. In FIG. 21f, the bobbit box cartridge 745 is shown installed in the barrel 759. The suture catcher rod 749 and needle 753 are also shown in FIGS. 21f and 21g. FIG. 21g depicts a cross-sectional view of the barrel 759 taken further down the barrel 759, away from the handle of the device (not shown) as compared to FIG. 21f. A guide slot 763 is shown in FIG. 21g and provides an area for the suture material (not shown) to pass.

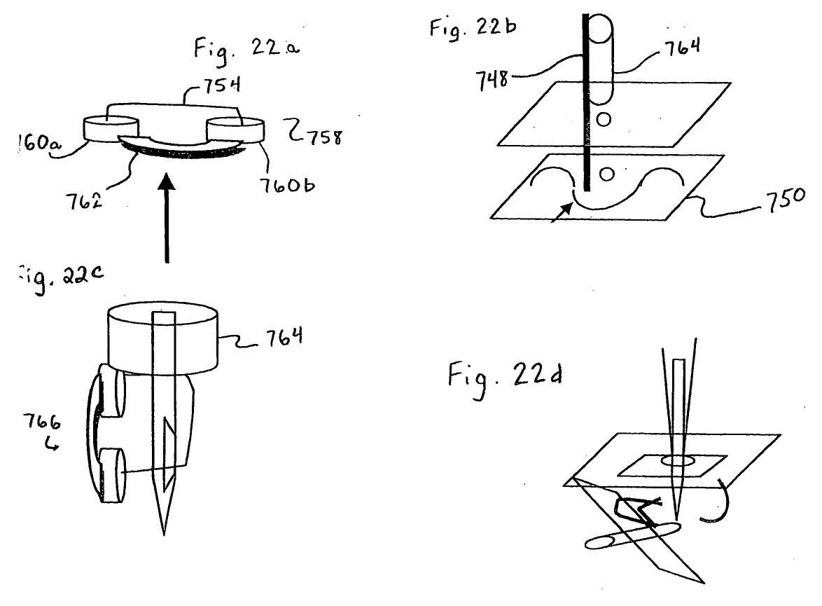

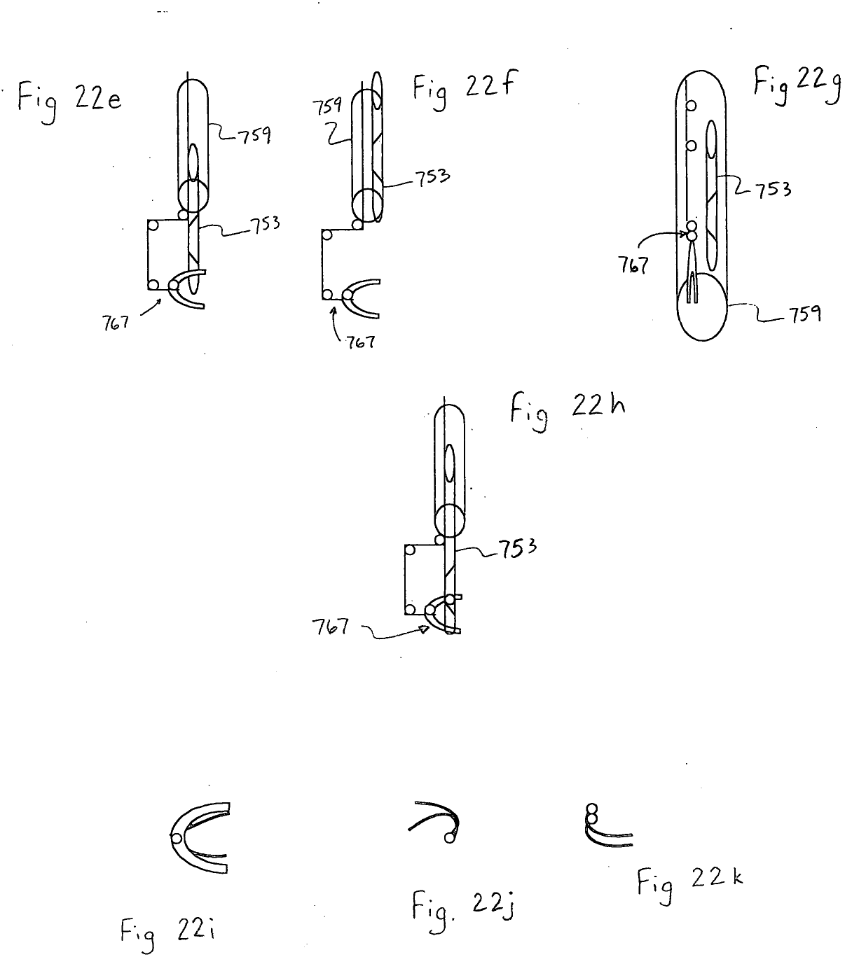

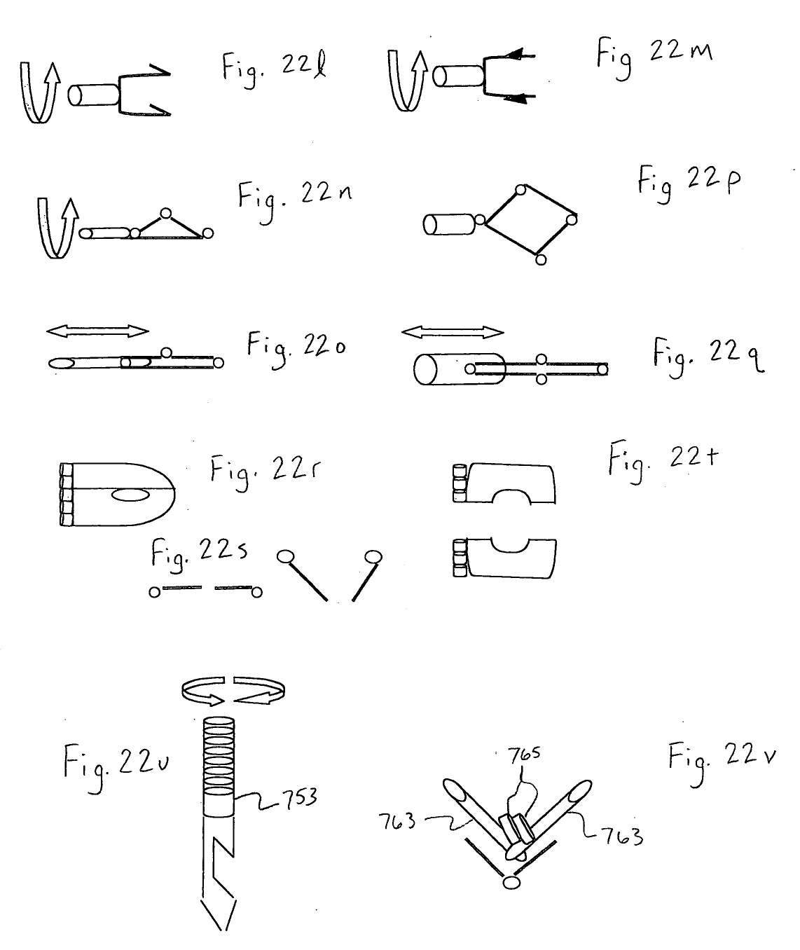

FIG. 22a depicts a bobbit-style suture holder device 758. The suture holder device 758 includes two bobbits 760a and 760b which hold the suture material 754. The suture material 754 can be drawn between the bobbits 760a and 760b from left to right or right to left. The bobbits 760a and 760b are interconnected by a hollow tube 762 containing suture material (not visible) and are also interconnected by external suture material 754, which is free to be caught by the needle.

FIG. 22b depicts a base 750 secured to the shaft 748. In this embodiment, the shaft 748 includes a needle guide 764. The bobbits 760a and 760b of FIG. 22b may be snapped into place in the base 750 thereby facilitating easy switching among different suture materials and suture sizes.

FIG. 22c depicts an arrangement similar to that of FIG. 22a, but with an external bobbit assembly 766. The external bobbit assembly 766 is optional and may be placed near the end of the needle guide 764.

FIGS. 22d-22v depict various embodiments of a suture catcher for use in accordance with certain embodiments of the invention. The suture catcher functions to hold the suture after the needle advances to the full extent (i.e. after it passes through tissue, or is reloading). The needle opens the bevel to release a suture it holds, or it can open the bevel to receive and then close to take the suture held by the suture catcher. In the embodiment shown in FIGS. 22e-h and 22v, the suture catcher is on a rod which may be triply-hinged and springed-loaded. When advanced, the rod forms a “C” shape to reach around the tissue, to meet the needle on the opposite side. When the needle is advanced by the user, at full advance it will rotate as shown in FIG. 22u and the bevel is then opened (for instance by pulling the trigger) and the suture catcher can then grasp the suture from the needle. Preferably, the needle penetrates the catcher in the final 0.5 to 0.2 cm of the full needle advance distance. Preferably, the catcher is similar to tissue, in that the needle easily penetrates and pushes the suture catcher open. This avoids any significant friction or force which might tear or rip the suture. The catches at the end of the suture catcher may be “U” shaped, see FIGS. 22i-k, or “U” shaped with barbs, see FIGS. 22l-m, or in the form of a closed loop or “0” shape, see FIGS. 22n-22t. In one embodiment of the closed loop shape, the catcher is hinged to fold closed, see FIGS. 22n-22q. Preferably the suture catcher is constructed so that when the needle retracts or is not present, the catcher spring hinge design closes the catcher. When the catcher shaft is retracted into the barrel of the device, the hinged joints are leveled to form a straight shaft. When the catcher is advanced the spring hinged elbow joints may form the open “C”, “U”, or “O” shape. In operation, for instance, the suture catcher of FIGS. 22n and 22p may be advanced out of the barrel with the advancing of the needle. With the needle bevel open and the needle withdrawn, the suture catcher may close on the suture, and in this way hold it.

With particular reference to the Figures showing the suture catcher embodiments, FIG. 22e shows the suture catcher 767 with the needle 753 advanced. FIG. 22f shows the embodiment of FIG. 22e with the suture catcher 767 advanced. FIG. 22g shows a similar embodiment in the closed position, with the suture catcher 767 and the needle 753 inside the barrel 759. FIG. 22h shows a similar embodiment, with the needle 753 advanced to release a suture to the suture catcher 767. FIGS. 22i-k show different positions of one embodiment of the suture catcher 767 as it goes from an open position, FIG. 22i, to a partially open position FIG. 22j, to a closed position, FIG. 22k. FIGS. 22n and 22o show a triangle-shaped suture catcher in an open and closed position, respectively. FIGS. 22p and 22q show a quadrangle suture catcher in an open and closed position, respectively. FIGS. 22r and 22t show a flat “hole” suture catcher in a closed and open position, respectively, and FIG. 22s shows side views of the flat “hole” catcher. FIG. 22u shows the circular movement action of the needle 753 which may be used to release a suture from an open bevel, and to reload a suture and close the bevel. Finally, FIG. 22v shows a close-up view of a hinge for a suture catcher, with a catcher shaft 763 and multiple springs 765.

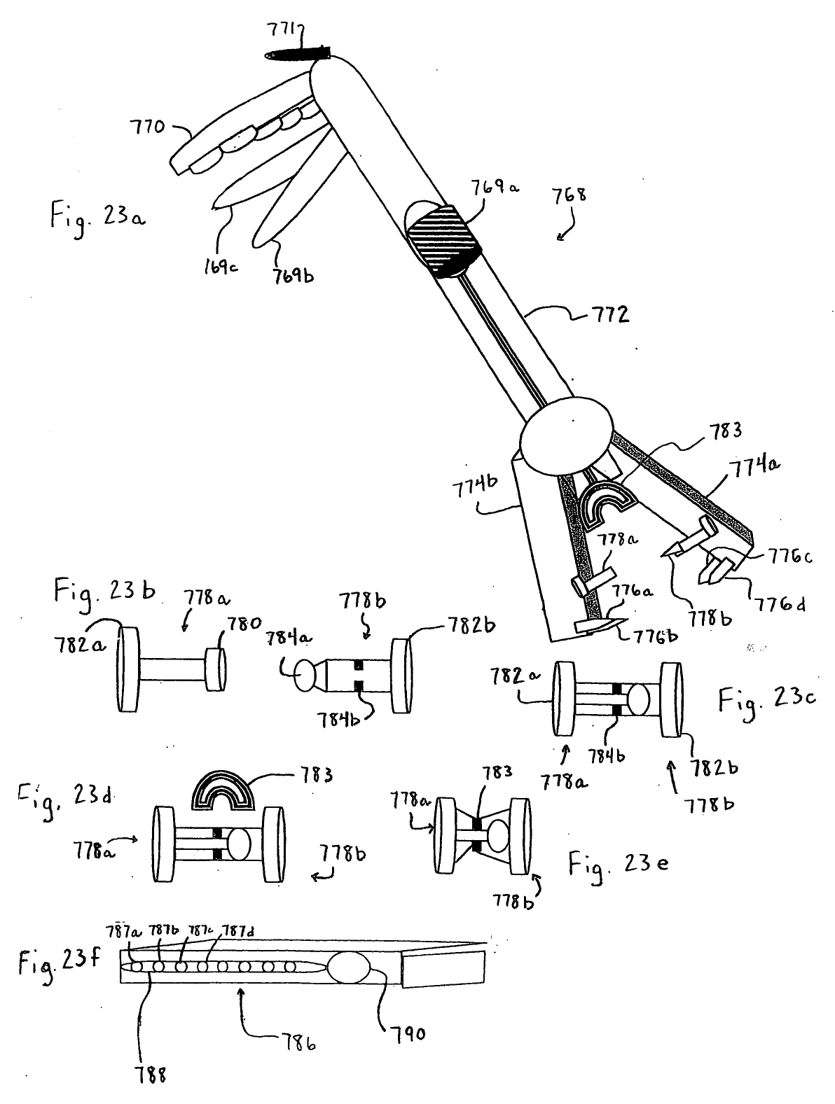

FIG. 23a depicts a rivet driver 768 in accordance with another embodiment of the invention. The rivet driver 768 is configured to drive and crimp a fastener across tissue. The rivet driver 768 may be used to fasten or close dense tissue such as fascia, or the diaphragmatic crus during esophageal hernia repairs. The rivet driver 768 may include a rotation knob 769a and certain control levers 769b and 769c to provide a user control over the disposition and operation of the rivet driver 768. The rivet driver 768 also includes a handle 770 attached to an elongated shaft 772. One of the control levers 769b may control, for instance, the closing of elongated members 774a and 774b which make up the rivet tool.

Each elongated member 774a and 774b may include one or more claws or barbs 776a-d. The barbs 776a-d are useful to help keep the tissue together or to keep the tissue in position prior to the rivet fastener or nail traversing the tissue. The barbs 776a-d may have sharp thin teeth (not shown). In one embodiment, the barbs 776a-d are very short, which allows easy withdrawal from tissue and avoids damage to the tissue grasped by the barbs 776a-d.

In one embodiment, the rivet driver 768 pushes the fastener, which is made up of two rivet portions 778a and 778b together so that the rivet portions 778a and 778b will not disengage once released by the rivet driver 768. A rivet portion 778a or 778b fits in each elongated member 774a and 774b. In certain embodiments (not shown) multiple rivet portions (not shown) may be stored in a channel or a cartridge assembly which automatically loads a new rivet portion upon releasing a used rivet portion. Alternatively, a nail be used instead of rivet portions 778a and 778b. A crimper button or lever 771 may be provided to allow a user to separately control the crimping of a rivet, with a crimper 783, as described below.

FIG. 23b depicts rivet portions 778a and 778b which may be constructed from non-absorbable material including stainless steel, nylon, or plastic, or an absorbable material such as polyglactic 910 (sold by Ethicon, Inc. under the tradename VICRYL), polydioxanone (sold by Ethicon, Inc. under the tradenames PDS and PDS II), chromic material, a polymer material such as polyethylene, or any other suitable material. Each rivet portion 778a and 778b is configured to be complimentary to and engage its complimentary rivet portion 778a or 778b. The rivet portion 778a on the left includes a narrow portion with a tip 780 which penetrates tissue. The tip 780 may be blunt, or bulbous, or sharpened. A flat, disc-shaped head 782a is also provided and helps to anchor the rivet portion 778a to the tissue the rivet portion 778a is driven through.

The partner rivet portion 778b has a similar diameter, but is slightly larger, in order to accommodate the complimentary rivet portion 778a. One end of the partner rivet portion 778b is open to provide access to a hollow tube 784a. Hollow tube 784a includes an internal indentation, notch or ring 784b which the tip 780 of the rivet portion 778a will engage once the rivet portions 778a and 778b are pushed together. This indentation 784b helps to prevent disengagement once the rivet portions 778a and 778b are pushed together. One end of rivet portion 778b may also be provided with a flat, disc-shaped head 782b.

FIG. 23c depicts a pair of rivet portions 778a and 778b which have been partially pushed together. In the configuration depicted in FIG. 23c, the rivet portions 778a and 778b are engaged and from this position, they may normally be more fully pushed together so as to form secured fastener. The position of the heads 782a and 782b of the rivet portions 778a and 778b is visible in this drawing as being useful to provide an anchor in the tissue the rivet portions 778a and 778b are driven through.

FIG. 23d depicts rivet portions 778a and 778b in a closed position. Additionally, a crimper 783 is provided for use with rivet portions 778a and 778b. The crimper 783 compresses the middle portion of the fastener formed by the rivet portions 778a and 778b, between the two heads 782a and 782b of the rivet portions 778a and 778b. The crimper 783 helps to assure the rivet portions 778a and 778b join solidly to form a secure fastener.

FIG. 23e depicts the rivet portions 778a and 778b in a closed position after application of the crimper 783 to secure the rivet portions 778a and 778b together. In this embodiment, the compression of the rivet portions 778a and 778b by the crimper 783 is clearly visible.

FIG. 23f depicts a rivet holder or cartridge 786 which may be fitted into a rivet driver such as rivet driver 768 shown in FIG. 23a. The rivet holder 786 may include a channel 788 with a riveting zone from which the rivets may be applied. The channel 788 holds the rivet portions 787a-d until they are ready for use. The channel 786 may also include an opening 790 which allows the rivet portions 787a-d to be released as they are used, one at a time. The rivet holder 786 may be configured to hold multiple rivets. In one embodiment, the rivet holder 786 is configured to hold up to 10 rivet portions 787a-d.

FIG. 23g depicts a two rivet portions 778a and 778b, which are similar to those described above, however in this embodiment internal fastener rivet portion 778a splits at an angle (preferably about ninety degrees) inside the receiving rivet portion 778b, when closed, as is shown in FIG. 23h. The split, caused by the mechanical compressive force of the device pushing the fasteners together is not reversible through mild to moderate traction force. Thus, the rivet portions 778a and 778b are held together in this fashion. FIG. 23i depicts various ways the rivet portion 778a might be constructed to split, for instance into halves, thirds or quarters. In another alternative, rivet portion 778a may be used alone, without a complementary rivet portion 778b, as shown in FIG. 23j. In this embodiment, the part of the rivet portion 778a that splits is used to secure tissue.

Table 1 below shows suitable rivet sizes for use in accordance with certain embodiments of the invention. These sizes refer to an assembled and compressed rivet.

TABLE 1
RIVET SIZES

| Gauge | Head Diameter (cm) | Length (cm) |
|---|---|---|
| 4 | 0.5-1 | 0.5-2 |
| 6 | 0.5-1 | 0.5-2 |
| 8 | 0.5-1 | 0.5-2 |
| 10 | 0.5-1 | 0.5-1.5 |
| 12 | 0.5-1 | 0.5-1.5 |
| 16 | 0.5-1 | 0.5-1.5 |
| 18 | 0.25-0.5 | 0.5-1 |
| 20 | 0.25-0.5 | 0.5-1 |

Additionally, rivets portions 778a and 778b may be provided in the following dimensions: a shaft length of 0.5 cm with a total length when closed of 0.7 cm. The diameter of the rivet head may be 0.4-0.8 cm and the thickness 0.1 cm. The tip head may be 0.3-0.5 cm with a hollow tube diameter of 0.1 cm and an inner ring diameter of 0.08 cm and an exterior diameter of 0.1 cm.

FIG. 24a depicts a needle driver 792 with a handle 794 and an elongated shaft 796 with a needle 798 mounted thereto. Numerous control elements 800a-d are provided to control operation of the needle driver 792. A bevel lock 802 may also be provided to close the bevel 804 on the needle 798. The bevel lock 802 may be operated by one or more of the control elements 800a-d, for instance a lever 800a and a button 800b may be used to open or close the bevel lock 802 over the bevel 804. Levers 800c and 800d may be used to advance the needle 798.

FIG. 24b depicts the needle 798 of FIG. 24a, with the bevel lock 802 in an open position.

FIG. 24c depicts a needle 806 similar to that shown in FIG. 24a, however the needle 806 of FIG. 24c has an inverted bevel 808. In FIG. 24c, the bevel lock 802 is shown in a closed position.

FIG. 24d depicts the needle 806 of FIG. 24c, with the bevel lock 802 in an open position.

FIG. 24e depicts various needles 810a-h for use in accordance with certain embodiments of the invention, particularly the needle driver of FIG. 24a and the suturing device of FIG. 21a. Needles 810a-810d all include a blunt tip, while needles 810e-h include a sharp tip, which may be either a smooth circular or a cutting type of tip. The needles 810a-810h also vary in some instances in the bevel type 812 of the particular needle 810a-810h. Needles 810a and 810e have a traditional open bevel 812. This allows suture material (not shown) to slide in or out of the needle's grasp. Needles 810b and 810f have an inverted bevel 812. This arrangement is similar to that of the open variety, however the edges of the bevel 812 are turned inward, giving the needles 810b and 810f a different grasp of suture material. Needles 810c and 810g have a pusher style bevel 812. In this arrangement the suture material is generally held in place while the needles 810c and 810g push through a tissue, and the suture material is left behind as the needles 810c and 810g exit the tissue. Needles 810d and 810h have a puller-style bevel 812. These needles 810d and 810h are similar the needles 810c and 810g, however they work in the opposite directions. Thus, needles 810d and 810h pull suture material through tissue as they exit the tissue and leave the suture material behind as the needles enter and push through tissue. Preferably the needles are constructed from stainless steel, however they may also be constructed from other suitable materials.

Where a needle is intended for use with the suturing device of FIG. 21a, the pusher-style and puller-style needles are particularly useful. When the suturing device uses an internal bobbit, the puller style needles 810d and 810h are useful. When the suturing device uses an external bobbit, the pusher style needles 810c and 810g are useful.

In certain embodiments, the needles 810a-810h are provided in the following dimensions: a needle gauge of 6, 8, 10, 12, 14, 16, 18, 19, 20, 22, 24, 28, 30, 32; a bevel length of from 0.25 cm to 1 cm; and an overall needle length of from 2 cm to 15 cm, with the portion of the needle intended to be internal to the device being from 1 cm to 10 cm and the portion of the needle external to the device ranging from 1 cm to 5 cm. Needles of other dimensions may also be used in accordance with the invention.

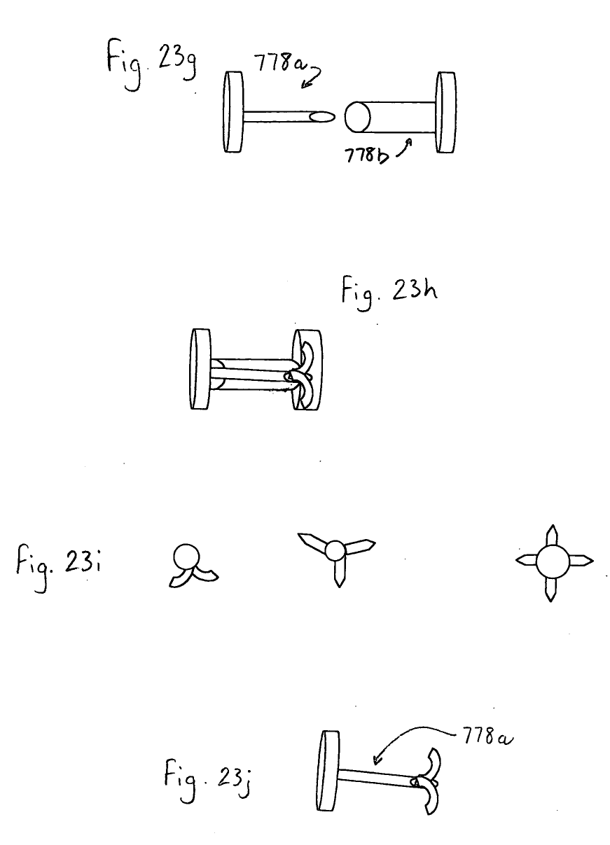

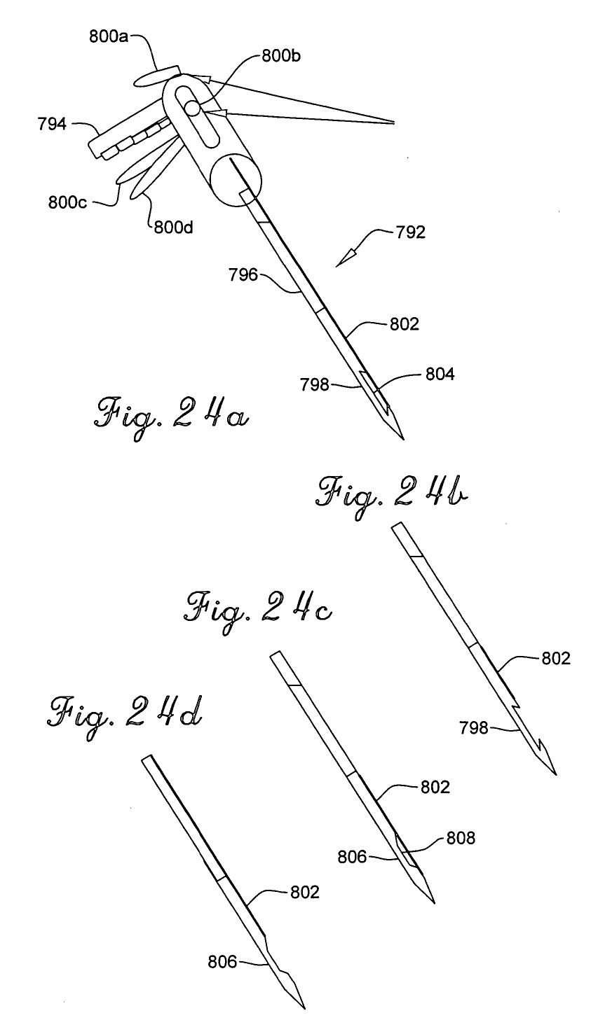

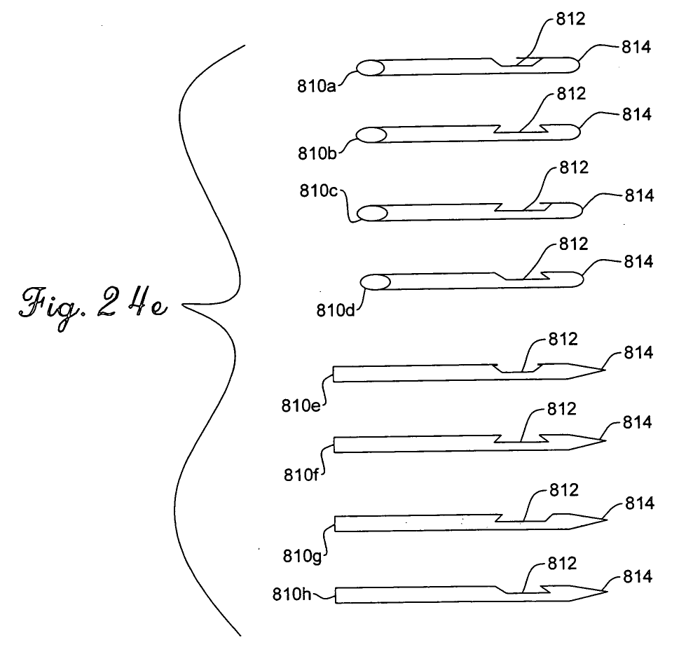

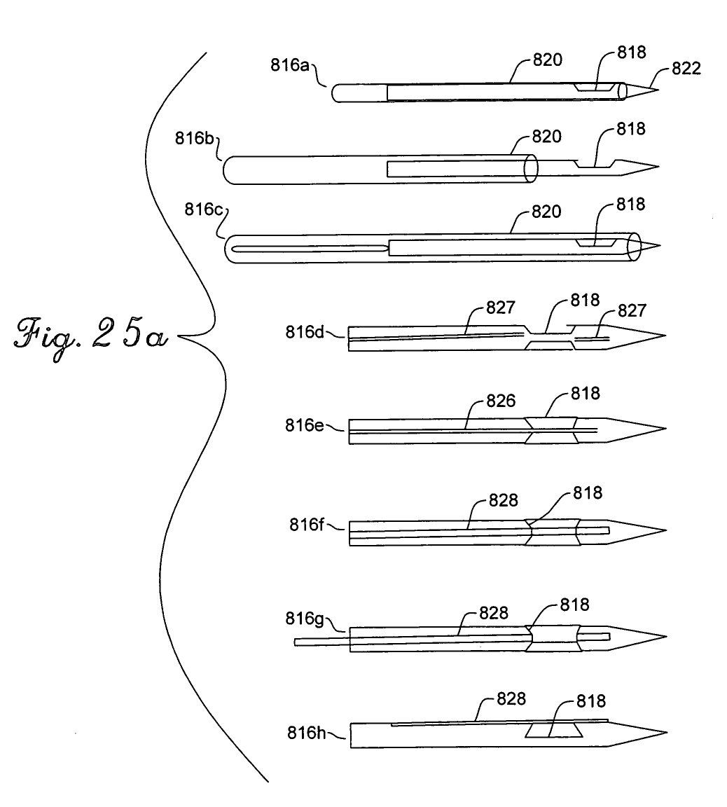

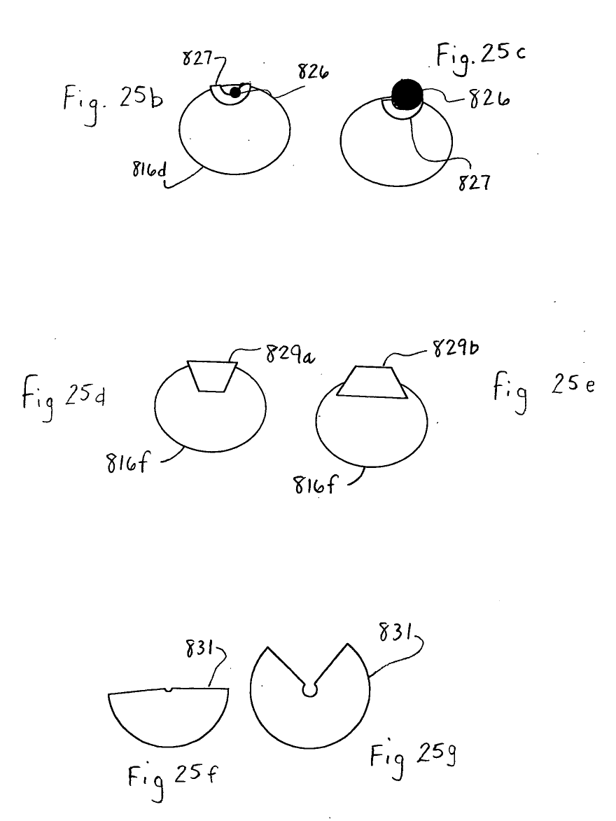

FIG. 25a depicts needles 816a-h for use with certain embodiments of the invention. These needles 816a-h include locking features for their bezels. Needle 816a includes a sheath 820 which is a hollow tube that slides laterally along the needle 816a to cover and close the bezel 818. In one embodiment, the sheath does not cover the tip 822 of the needle 816a. The sheath 820 may also slide along the needle to an open position, as is depicted with needle 816b. A needle 816c may also be provided with a pusher or mounting rod 824. In this embodiment, various needles are exchangeable and may be mounted to the pusher 824 as desired by the user. Alternatively, the needle and the pusher 824 may be fused together.

A needle 816e may also be provided with a bevel lock mechanism incorporating a wire 826. The wire 826 may slide from an open position to a closed position over the bevel 818. As can be seen in with respect to needle 816e, the wire serves to partially close access to the bevel 818, thereby preventing any suture material from entering or leaving the bevel 818, as the case may be. A wire guide channel 827 may be provided in which the wire 826 may at least partially rest.

A needle 816f may alternatively be provided with a bevel lock mechanism based on a slidable plate 828. Needle 816f is shown with a slidable plate 828 in a closed position. Needle 816g is shown with a slidable plate 828 in an open position, away from the bevel 818. Needle 816h is depicted from a lateral view and is shown with a slidable plate 828 in a closed position over the bevel 818.

FIGS. 25b and 25c depict a cross-sectional view of a needle 816d which uses a wire-type bevel lock. In FIG. 25b, the cross-section is taken above the bevel. A wire guide channel 827 is shown which may be used to provide a guide space for the wire 826. FIG. 25c shows a cross-sectional view similar to that shown for FIG. 25b, however in FIG. 25c, the wire 826 sits partially above the wire guide channel 827 profile.

FIGS. 25d and 25e depict a cross-sectional view of the needle 816f with a slidable plate-type bevel lock. As may be appreciated from the drawings, different types of slidable plates 829a and 829b may be used to lock the bevel on the needle 816f. These needles are preferably provided with a channel in which the slidable plate 829a and 829b may slide. FIGS. 25d and 25e show this channel at the intersection of the slidable plate 829a or 829b and the needle 816f.

FIGS. 25f and 25g depict a cross sectional view of the bevel portion of the needle 831. In FIGS. 25f and 25g, the cross section is taken at the bevel, and the remaining raised portion of the needle 831 is not shown, thus the view is only a cross section of the bevel portion of the needle 831. The bevel portion may a variety of sizes, as is useful for the particular application. By way of example, FIG. 25f depicts a bevel portion with approximately 50% of the needle 831 cut away, FIG. 25g depicts a bevel portion with approximately 20% of the needle 831 cut away.

Thus, it may be appreciated that the needle may be fixed relative to the device, and the needle bevel is opened or closed by a mobile lock such as the sheath, plate or wire lock described above. Alternatively, the needle may be mobile and the needle bevel lock mechanism, such as the sheath, plate or wire described above, may be in a fixed position relative to the device. The needle bevel is constructed so that it may hold a suture or be empty or release a suture.

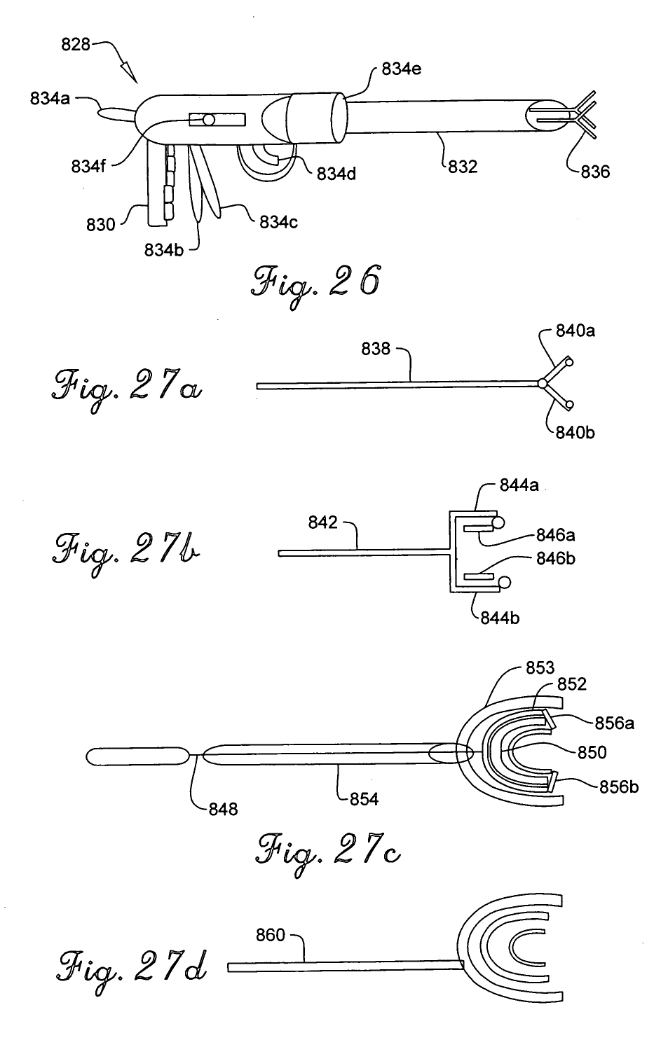

FIG. 26 depicts a pistol-style ligation device 828. The device 828 includes a handle 830 associated with an extended shaft 832. Certain control elements 834a-f are provided to allow a user control over the operation of the device 828. In one embodiment, the device 828 has at least one of four distinct features. First, the device 828 may grasp two sutures to be joined as is commonly done with an open instrument. Second, the user may position the device 828 and choose the desired tension of the sutures and the actual site of the binding of the suture material. Third, the equivalent of a knot may be formed by fusion of the suture material either thermally or through adhesive fusion. Finally, the device 828 may provide the option of cutting off the sutures above the fusion or adhesive knot. Alternatively, the cutting may be performed by a separate scissor device (not shown).

One of two types of adhesive ligatures are preferably used in accordance with certain embodiments of the present invention such as that depicted in FIG. 26. One type involves the injection of liquid adhesive into a ligature forming mold (not shown) which may be heat sensitive. Upon application of heat, the liquid adhesive hardens to form a permanent to semi-permanent ligature, depending upon the material being used. The adhesion may be accomplished with a variety of suitable agents, including mixtures of multiple agents. Alternatively, mixtures of reactive agents may be formed which undergo a chemical reaction, either in the presence of heat energy or not, resulting in a permanent or semi-permanent solid with the sutures bound together therein. Ligatures may also be formed using an adhesive that hardens upon contact with some third substance, which third substance may act as catalyst. In one embodiment, the adhesive hardens upon contact with a mold frame. Suitable adhesives include plastic, polymeric silicon (available from Dow Chemicals under the tradename SILASTIC), polypropylene, polyglycolic, polyvicryl, GORTEX, cellulose, a chromic material, polyethylene, ceramic, glass, and stainless steel. In another embodiment, sutures are bound using a staple configuration

Control element 834d is a trigger that activates a scissor or blade to cut the suture material above the fusion site. In one embodiment, the suture material is cut 0.05 cm-1.0 cm above the fusion site. Preferably, control element 834d causes the blade or microscissor or regular scissor to advance to a position just above the suture so that the excess suture material may be cut away. Preferably the blade or scissors is advanced and then automatically retracted as a result of the single trigger pull.

The ligation device 828 may also include one or more rods 836 which are useful for manipulating the sutures. For instance, the rods 836 may be used to push or grasp or pull the sutures so that they may be ligated together. These rods 836 enable the ligation device 828 to be used to securely grasp the suture material the user desires to ligate, tie, or fuse together. The rods 836 may be integrated with the extended shaft 832. In one embodiment (not shown) there are three rods 836 provided with the ligation device 828. One rod is for grasping two sutures, one rod is for fusing or adhesive ligation stapling, and the third rod may be used for cutting the suture after ligation. Alternatively, a combination of a lesser number of the aforementioned rods may be provided.

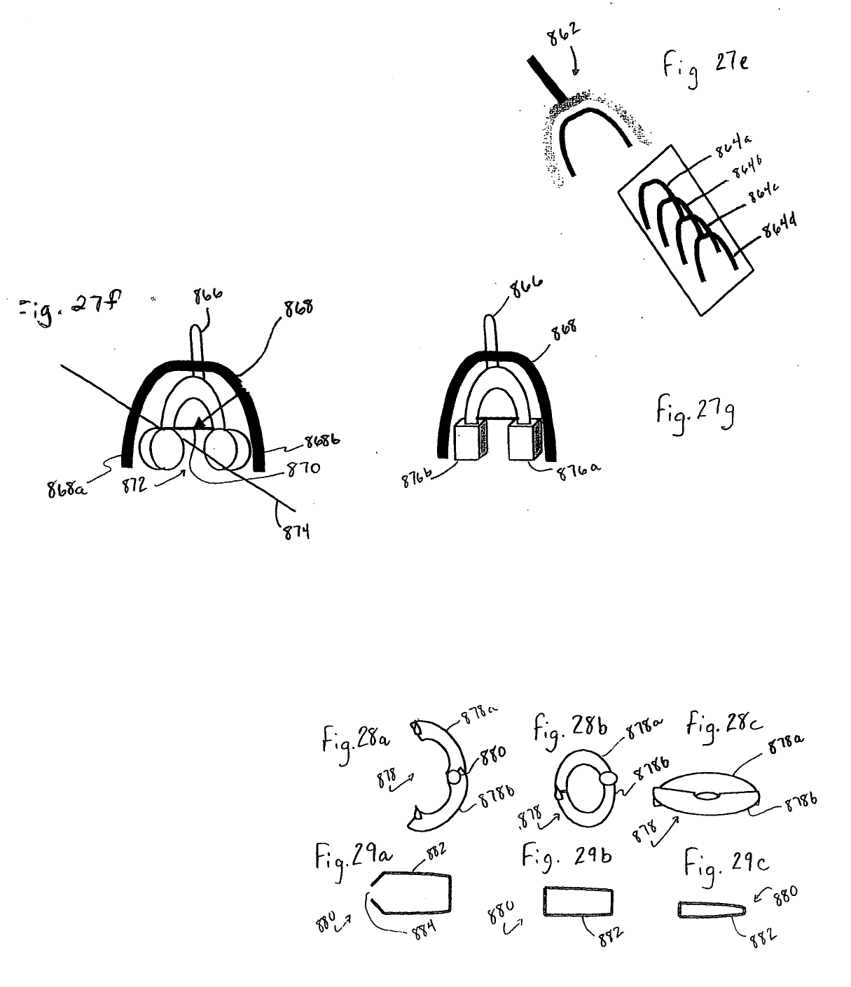

FIG. 27a depicts one embodiment of a grasping rod 838 for use in accordance with one embodiment of the invention. The grasping rod 838 includes two elongated grasping elements 840a and 840b, each of which are configured to close about a suture or other material and grasp it for manipulation by a user. In this embodiment, an adhesive substance is loaded in to a ligation device such as ligation device 828 depicted in FIG. 26. The adhesive substance may be provided in a particular shape or configuration, for instance in the configuration of a staple. The adhesive substance is applied or compressed on to the suture material which needs to be bound and the adhesive substance fuses together with the suture material. The adhesive substance may be fused by the activation of a fusion switch. The fusion switch activates a temperature controller which heats a heating element to cause fusion of a temperature sensitive adhesive.

FIG. 27b depicts a stapler 842 for use in accordance with an embodiment of the invention. This stapler 842 may be used to staple two sutures together instead of using a knot to tying them together. The stapler includes elongated grasping elements 844a and 844b, similar to that depicted in FIG. 27a. Each of the elongated grasping elements 844a and 844b is also provided with a thermal element 846a and 846b which may be used to induce melting of suture material together. In one embodiment, the thermal element is switched on by the touching of the thermal elements 846a and 846b together which closes an electrical circuit. This may then either melt the suture material or initiate a heat sensitive reaction with some other binding compound (as described below). Preferably this heat need only be applied for a few seconds, after which the thermal element 846a and 846b may be released Alternatively, the thermal elements 846a and 846b may be replaced with binding compound. The binding compound may take on any suitable form. In one arrangement, the binding compound is a non-metallic material which is pressed onto the sutures to be joined and the non-metallic material adheres to the sutures, creating a bond which is the functional equivalent of a knot tying the sutures together. The bond may be formed as a result of chemical reactivity between different binding compounds being brought together, or it may be spontaneous, as in the case of cyanoacrylate based glues, or a heat sensitive material such as chromic, plastic, nylon. The material may or may not be absorbable. Thus, the grasping rod 842 may be used to grab the sutures that are to be fastened together and then secure them through a thermal fusion action by use of the thermal elements or with binding compound.

FIG. 27c depicts a manipulation rod 848 for use in accordance with an embodiment of the invention. This rod 848 is configured to grasp the sutures together, and then apply an injectable adhesive which binds the sutures together. A variety of suitable configurations may be used in order to apply the adhesive to the sutures. In the embodiment depicted in FIG. 27c, the adhesive 850 is encased within an injectable mold 852. In use, the mold 852 is compressed by lateral movement of the rod housing 854, or alternatively movement of the rod 848 within the rod housing 854, which compresses the mold 852, forcing the adhesive 850 out each end of the adhesive housing 856a and 856b so that the adhesive 850 is applied to the sutures. Alternatively, the mold 852 may be opened to grasp the sutures to be bound together. At the desired position, the adhesive is injected and the sutures become bound. Preferably, the glue or adhesive bonds almost instantly so that after application of the glue or adhesive to the already-present sutures, they may be released from the grasp of the device and they are bound together. Further, a container 853 may be provided to hold and dispense the glue or adhesive for the stapler. The container 853 may be replaceable and tube-shaped and preferably dispenses the glue or adhesive as a result of pressure applied to the outer surfaces thereof.

FIG. 27d depicts an adhesive ligation staple rod 860 similar to that shown in FIG. 27c, but without the injector components. This staple rod 860 can be used to secure sutures together with an adhesive staple. In use, the staple rod 860 can be pushed into an open position followed by closure around the sutures which are to be secured together. An adhesive may then be applied and the sutures thereby secured together.

FIG. 27e depicts an adhesive stapler loading device 862. The adhesive stapler loading device 862 is configured to accept multiple staples 864a-864d of different types. In on embodiment, two types of staples are provided, either hot or cold staples. The cold staples are compressed around the sutures to be fastened together, and no thermal energy is required to secure the ligation. The hot, or thermal staples, are configured to be compressed around the suture and thermal energy is applied to precipitate a chemical reaction to secure a bond between the sutures being fastened together. Preferably, the staples are nonmetallic, for instance they may be configured from nylon, polypropylene, polyethylene, or another plastic or other suitable material which may be used to bind the sutures together with either or both of compressive force and heat.

FIG. 27f depicts an injector 866 which injects an adhesive substance into a compressor mold 868. The compressor mold 868 may be either spherical, or rectangular, or box shaped. A suture guard 870 is provided to capture the suture material in an adhesive compression chamber 872 within the compressor mold 868. Once the suture material 874 is captured within the compression mold 868, two sides of the compression mold 868a and 868b are approximated, or closed together, and the adhesive is injected. In this way, the suture material is glued or bound together.

FIG. 27g depicts an injector 866 similar to that shown in FIG. 27f. The injector 866 of FIG. 27g includes a compressor mold 868. However, this compressor mold 868 includes rectangular or box shaped compression features 876a and 876b. The compression features 876 and 876b may be provided in any of a variety of shapes or configurations. For instance, the circular spheres depicted in FIG. 27f or elliptical shapes or the rectangular shape shown in FIG. 27g. The spherical delivery of the adhesive results, from the device shown in FIG. 27f, results in a generally spherical shape of adhesive. The cube shaped delivery of the adhesive results, from the device shown in FIG. 27g, in a generally cube-shape of adhesive.

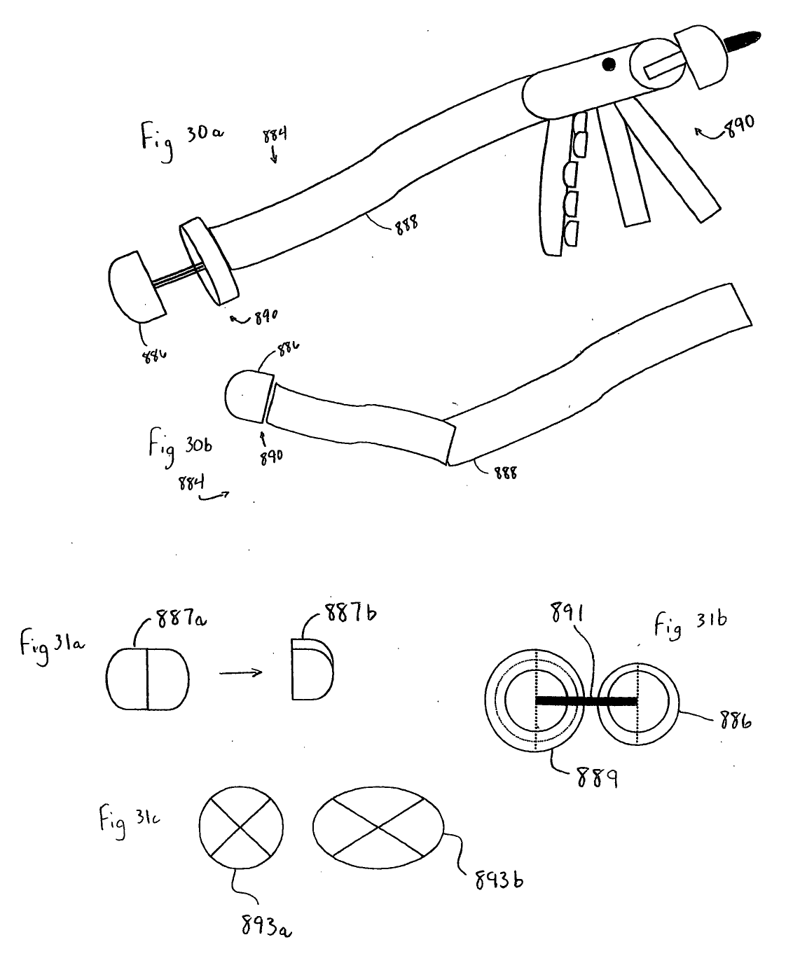

FIG. 28a depicts a tie clasp 878 which may be used to secure or bind sutures. The tie clasp 878 is configured from two semicircular shaped elements 878a and 878b which are pivotally attached to rotate about axis 880.

FIG. 28b depicts the tie clasp 878 of FIG. 28a in a closed position. In the position shown in FIG. 28b, semicircular elements 878a and 878b are closed so as to form a single circular, elliptical, or oval-shaped unit.

FIG. 28c depicts another drawing of the tie clasp 878 of FIG. 28a. In FIG. 28c the tie clasp 878 is closed tightly. As can be seen in the drawing, the semicircular elements 878a and 878b are closed so as to tightly bind any suture material that might be on the interior of the closed shape formed by the two circular elements 878a and 878b.

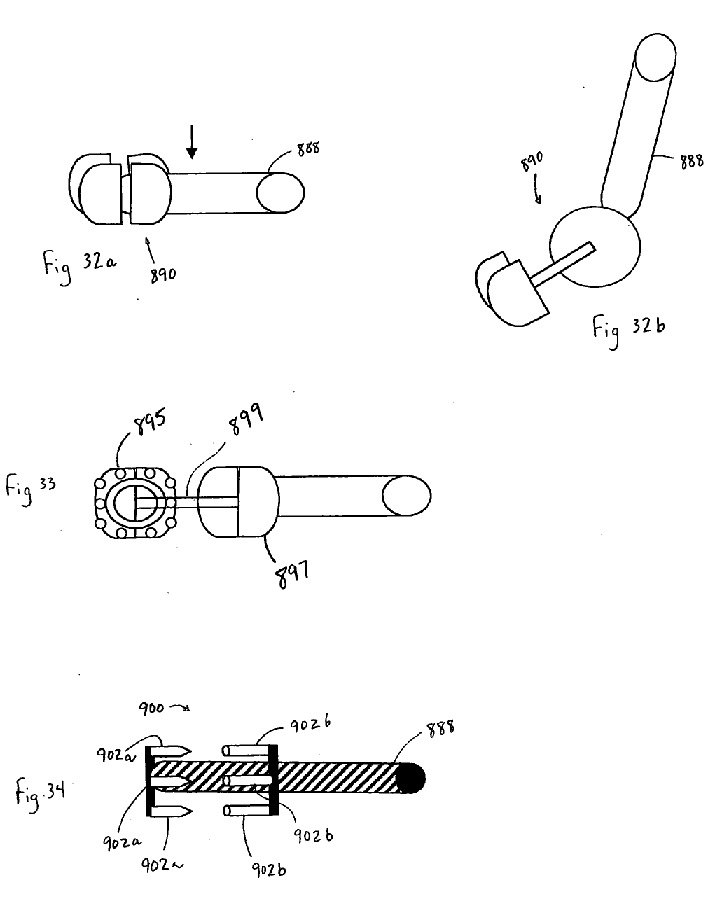

FIG. 29a depicts a clip 880 similar to that depicted in FIG. 28a. However, the clip 880 of FIG. 29a more closely resembles a staple than a device with two elongated semicircular rounded elements which pivot about an axis. The clip 880 of FIG. 29a may be constructed from a single length of wire, flat metal, or other suitable material 882. This single length of material 882 may be bent into what is generally a “U” shape with an opening 884 at one end.

FIG. 29b depicts the clip 880 of FIG. 29a in a closed position. The single length of material 882 is pressed together so that the opening 884 (depicted in FIG. 29a) is closed. Preferably the single length of material 882 is closed about one or more sutures so as to bind them together.

FIG. 29c depicts the clip 880 of FIG. 29a in another closed position. In FIG. 29c the clip 880 is closed tightly so that the single length of material 882 forms a loop with an interior area which is smaller than that depicted in FIG. 29b. This may be useful in order to bind one or more sutures together tightly.

Referring to FIGS. 28a-28c and 29a-29c certain clasps 878 and 880 are shown which may be used with certain embodiments of the present invention. These clasps 878 and 880 are configured to have an opening which may accept the one or more sutures to be bound. The clasps 878 and 880 are then closed about the sutures so as to bind them together. The clasps 878 and 880 may be closed in either loose or tight positions depending on the wishes of the user and upon the tissue response. Alternatively, the clasps 878 and 880 may be useful for binding multiple tissues or closing off openings in tissue, as in the case of closing a severed bowel, vessel, or other tubular-shaped tissue, or tissue with an opening.

FIG. 30a depicts a circular stapler device 884 in accordance with an embodiment of the invention. The circular stapler device 884 is configured to staple pieces of bowel (stomach, colon, intestine, esophagus, or other tubular tissue) together. The circular stapler device 884 is useful to anastomose, or interjoin multiple tissues, such as tubular vessels. The circular stapler device 884 may be configured in a specific inner anastomosis diameter with a minimum of from a minimum of about 1 cm to about 3 cm. The circular stapler device 884 is configured to collapse into a smaller diameter when necessary to enter a laparoscopic port, or the bowel, prior to closure for stapling. The circular stapler 884 includes an anvil 886 which is attached to an elongated portion 888 and is operated by a handle configuration 890. The handle configuration 890 is similar to that provided with respect to other embodiments of the invention (as previously described). The stapler head 890 is configured so that it may be angled in relation to the elongated shaft 888 as desired by the user. Angling the circular staple head 890 provides significantly improved positioning in a constricted space, as is commonly encountered during minimally invasive medical procedures.

FIG. 30b shows the circular stapler device 884 of FIG. 30a. However, the elongated shaft 888 is in an angled position. As described previously, when angled, the extended shaft 888 may facilitate better positioning of the stapler head 890. Also visible in FIG. 30b is the anvil 886 which has been positioned more closely to the extended shaft 888 than is shown in FIG. 30a.

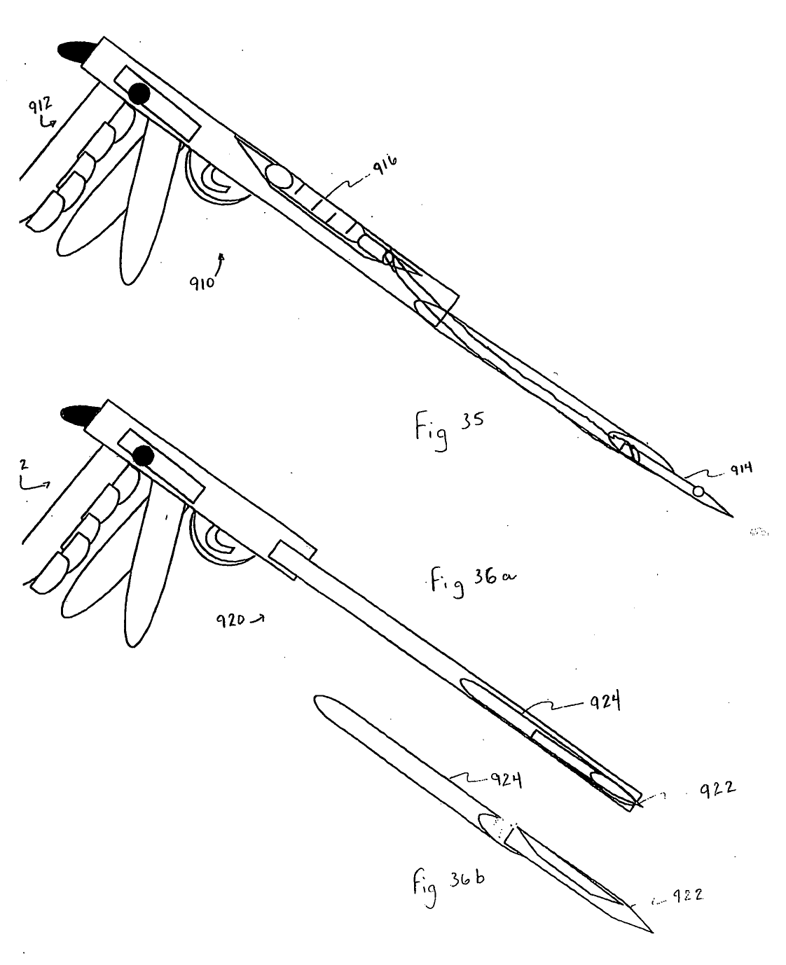

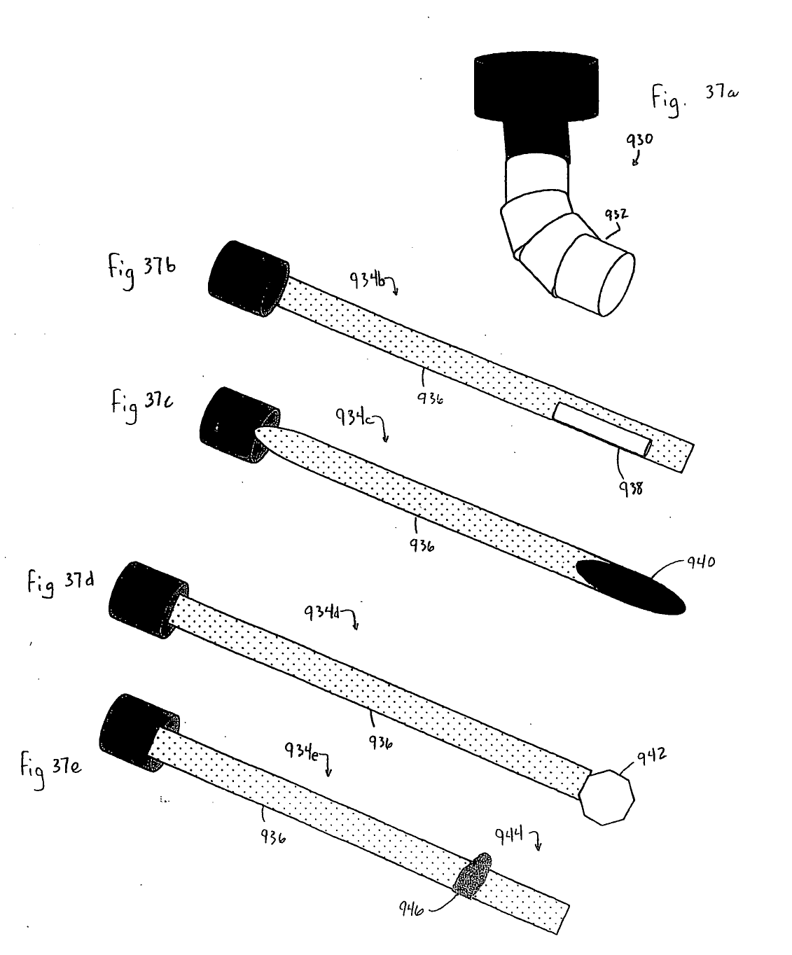

FIG. 31a depicts an anvil 887a for a circular stapling device such as that shown in FIG. 30a. The anvil 887a of FIG. 31a is hinged so that it may fold into a smaller size. In its folded form the it resembles anvil 887b. FIG. 31b depicts an anvil 886 and a base 889 connected by a rod 891. FIG. 31c shows an anvil 893a with a quartered “pie” construction which allows for a size reduction (anvil 893b). The anvils shown in FIGS. 31a-31c represent methods of anvil construction that facilitate temporarily reducing the profile or size of the anvil so that it may be passed through skin or fascia through an entry port and for small enterotomy in gastric tissue, the small bowel or colon.

Referring to FIGS. 32a and 32b, the circular staple head 890 is depicted along with the extended shaft 888. In FIG. 32a the circular staple head 890 is shown essentially as a linear extension of the extended shaft 888 such that the circular staple head 890 is in line with the extended shaft 888. In FIG. 32b the circular staple head 890 is shown at an angle of approximately 45° with respect to the extended shaft 888. As discussed previously, this movement of the circular staple head 890 from a straight to an angled position at the desire of the user is useful in positioning and articulating the circular staple head 890 to anastomose tissues or other internal materials.

FIG. 33 depicts an anchor face with a staple ring and anchor stud needles. The needles pass through the tissue and enter the hollow needle pockets prior to stapling. FIG. 33 shows the stapler guide receptacle to close the staples after they penetrate the tissue being anastomosed. Both the anvil 895 and the base 897 are shown “flat faced”, that is, in line with the connecting rod 899, so as to facilitate passage of the device through a laparoscopic port.

FIG. 34 depicts a side view of a portion of anchor 900 showing the anchor stud needles 902a and the hollow needle pockets 902b. The needles 902a pass through tissue and enter the hollow needle pockets 902b so as to ensure the tissue is grasped by the device and properly held in place. The needles 902a may also help to ensure proper position of the tissue for adequate stapling. The extended shaft 888 of the device is also shown in FIG. 34.

FIG. 35 depicts an aspiration or injection device 910. The aspiration or injection device 910 includes a handle portion 912 similar to that described with respect to other embodiments of the invention, see, for instance, FIGS. 10b, 23a, 24a, and 26, among others. The aspiration or injection device 910 may be provided with a needle 914 that may be manipulated, and/or operated by one or more of the numerous control devices provided with the handle 912. The needle 914 may operate in connection with a syringe 916 which is preferably removable. In this way, material in the syringe 916 may be injected through the needle 914 into a patient. Alternatively, material from the patient may be withdrawn through the needle 914 and into the syringe 916. In one embodiment the needle 914 is removable and may be replaced by any one of a variety of different tools, including needles of varying sizes and/or materials. In one embodiment, the needle may be manipulated by the trigger. For instance, the needle 914 may be advanced and/or retracted by operation of one or more trigger pulls.

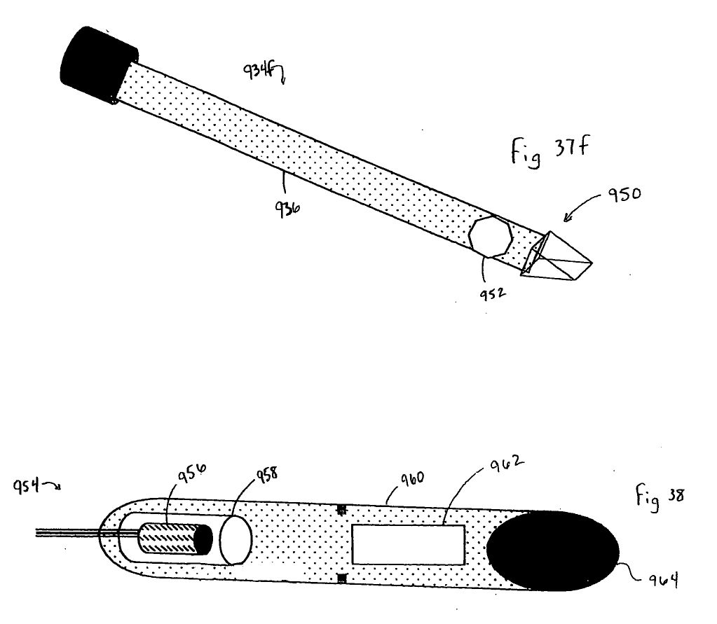

FIG. 36a depicts a biopsy device 920. The biopsy device 920 is provided with a handle 912 configured like the handles of the previous embodiments, for instance, like the handle of FIG. 35. The biopsy device 920 is provided with a biopsy needle 922 which is arranged so that it may be advanced and/or withdrawn as required by a user. A protective sheath 924 is also provided. The protective sheath 924 serves to seal off the sample containing portion of the biopsy needle 992. The biopsy device 920 is configured to safely obtain a biopsy sample with a needle and at the same time avoid the risks associated with obtaining biopsy samples via transcutaneous needles, which are known to leave a needle track that may be at least partially filled with biopsy material in the subcutaneous tissue or skin. In certain instances, this may result in cancer and/or infection or the subcutaneous tissue or skin.

In operation, the sheath 924 is retracted so that the needle 922 is outside of the biopsy device 920 and the needle 992 is inserted into the tissue to be sampled. At this point, the needle 922 is beyond the sheath 924 as is shown in FIG. 36b. Subsequently, the sheath 924 is advanced to cover the needle 922 and effectively protect the biopsy material collected within the needle 922 from all other tissues. The entire biopsy device 920 may then be withdrawn from the patient, thereby safely removing the biopsy material.

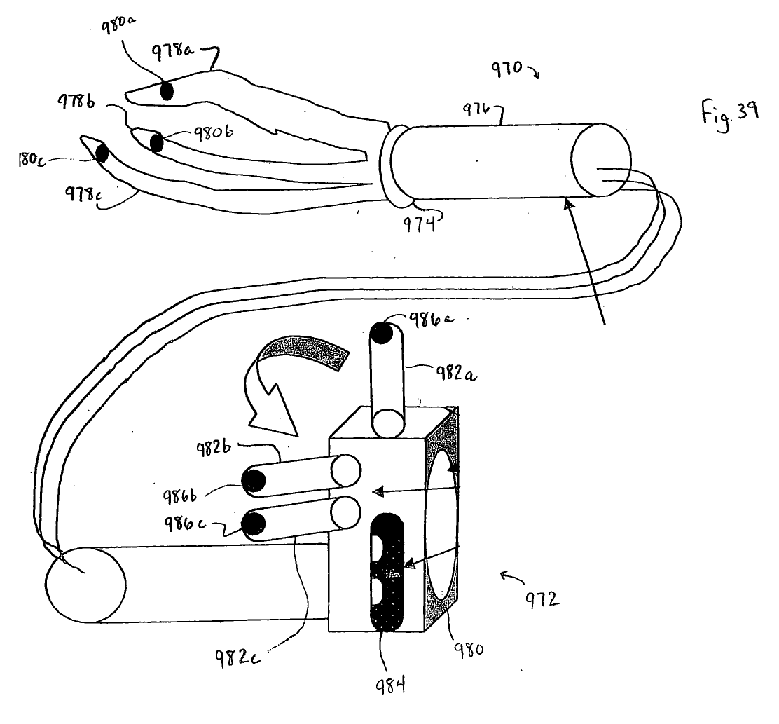

FIG. 37a depicts an external radiation machine 930 with a flexible connective elbow 932. The external radiation device 930 is useful for internal delivery of radiation therapy, especially gamma radiation. FIGS. 37b-37e depict various radiation delivery scopes 934b-934e for use in combination with the radiation machine 930 of FIG. 37a. The radiation scopes 934b-934e all include an elongated shaft 936 which is adapted for internal use on a patient. The radiation scopes 934b-934e vary in the type of radiation that they deliver and the way that that radiation is delivered.

FIG. 37b depicts a radiation scope 934b with an elongated shaft 936, which includes a window 938 for the delivery of radiation. Preferably the radiation scope 934b of FIG. 37b is configured to deliver gamma radiation.

The radiation scope 934c of FIG. 37c includes an elongated shaft 936 with an open end 940. The open end 940 is configured to deliver radiation from the radiation scope 934. Preferably, the radiation scope 934c of FIG. 37c is configured to deliver gamma radiation.

The radiation scope 934d of FIG. 37d includes an elongated shaft 936 with an open end 942 which is configured to allows for the emission of radiation. The radiation scope 934d of FIG. 37c is preferably configured for use with beta radiation.

The radiation scope 934e of FIG. 37e includes an elongated shaft 936 and also includes an enhanced tip 944, which allows for positioning. This is useful, for instance, when the radiation scope 934e is being employed for work on an inner-organ tumor. The radiation scope 934e may be used in conjunction with a separate imaging device which provides a visual field either through the use of visual image capture configuration or an ultrasonic image capture device. In the embodiment depicted in FIG. 37e, an ultrasonic tip 946 is provided in conjunction with the radiation scope 934e so that a single device may be used to both deliver a radiation treatment to a patient and to image the area of treatment. This may make the use of the radiation scope 934 easier, and lessens the invasiveness of the procedure for the patient. Alternately, the ultrasonic tip 946 may be associated with the radiation device 934e in another way. For instance, the ultrasonic tip 946 may be placed inside or on one end of the elongated shaft 936 of the radiation device 934e.

FIG. 37f depicts a radiation tool 934f in accordance with the invention. The radiation tool 934f includes an extended shaft 936 and a boring tip 950. The boring tip 950 allows placement of the tool into a patient's organ by boring through the surrounding tissue. Once in position, the boring tip 950 retracts to expose radioactive material 952 which is positioned near one end of the radiation tool 934f so as to enable radiation therapy for the patient.

FIG. 38 depicts an radiation delivery device 954 which is loaded with a radioactive emitter 956. The radioactive emitter may be a gamma or a beta emitter. The total dose for use in a given therapy is determined by the user, typically a radio-oncologist, or a radiation scientist prior to loading of the radioactive emitter 956. The radioactive emitter may be loaded within its own case 958, which is located within the scope 960 itself. The case 958 for the radioactive emitter 956 may be a lead shield. Preferably the radiation scope 960 is placed in a proper position within the patient prior to beginning the radiation therapy and all personnel in the room are evacuated. Once radiation therapy is to begin, the shield or case 958 is opened or moved so as to expose the radioactive emitter 956. The patient may be irradiated through either the window 962 or an end opening in the radiation scope 960. Once therapy is complete, the shield 954 is replaced to avoid irradiation of health care personnel.

FIG. 39 depicts a medical device 970 which includes a glove handle 972 similar to that depicted in FIG. 6. The glove handle is connected to a tool 974 which includes an elongated shaft 976. At one end of the elongated shaft 976 are three elongated finger members 978a, 978b and 978c. Each of the finger members 978a, 978b, and 978c are configured to mimic the motion and responsiveness of human fingers. In particular, finger member 978a replicates a thumb. Finger member 978b replicates an index finger. Finger member 978c replicates a middle finger. Each of the finger members 978a, 978b and 978c are provided with a pressure sensor 980a, 980b and 980c. The pressure sensors 980a-980c operate similarly to those previously described.

The glove handle 972 includes an opening 980 that allows a user to place his or her hand inside of the glove handle 972. Separate pockets may be provided for one or more of the fingers. In particular, a thumb pocket 982a, an index pocket 982b, and a middle finger pocket 982c may be provided as part of the glove handle 972. The fourth and fifth digits of a user's hand may be used to wrap around a grip rod 984 provided within the glove handle 972. Pressure sensors may also be provided as part of the finger pockets 982a, 982b and 982c. Pressure sensors 9862, 986b and 986c operate in a manner similar to that previously described. The glove handle 972 may be provided in either a left or a right-handed version for the respective hands of a user. Accordingly, the tool 974 is configured to mimic the hand configuration provided in the glove handle 972.

The medical device 970 as shown in FIG. 39 effectively extends the function of the right or left hand of a user from outside the patient to inside the patient. The finger members 978a, 978b and 978c are flexible and include pressure sensitive pads 980a, 980b and 980c to communicate, among other things, the tissue density and firmness of the grip by the tool 974. In one embodiment, the tool 974 is passable through an port of approximately 2 cm in diameter. Once inside the patient the tool 974 may open so that the finger members 978a, 978b and 978c are of a size of up to about the size of three average adult human fingers. In one embodiment, the finger members 978a, 978b, and 978c are configured not only to grasp or release an object but also to move up and down or laterally right to left. Further, the finger members 978a, 978b and 978c may be configured to rotate either individually or as a unit and also to move forward and backward.

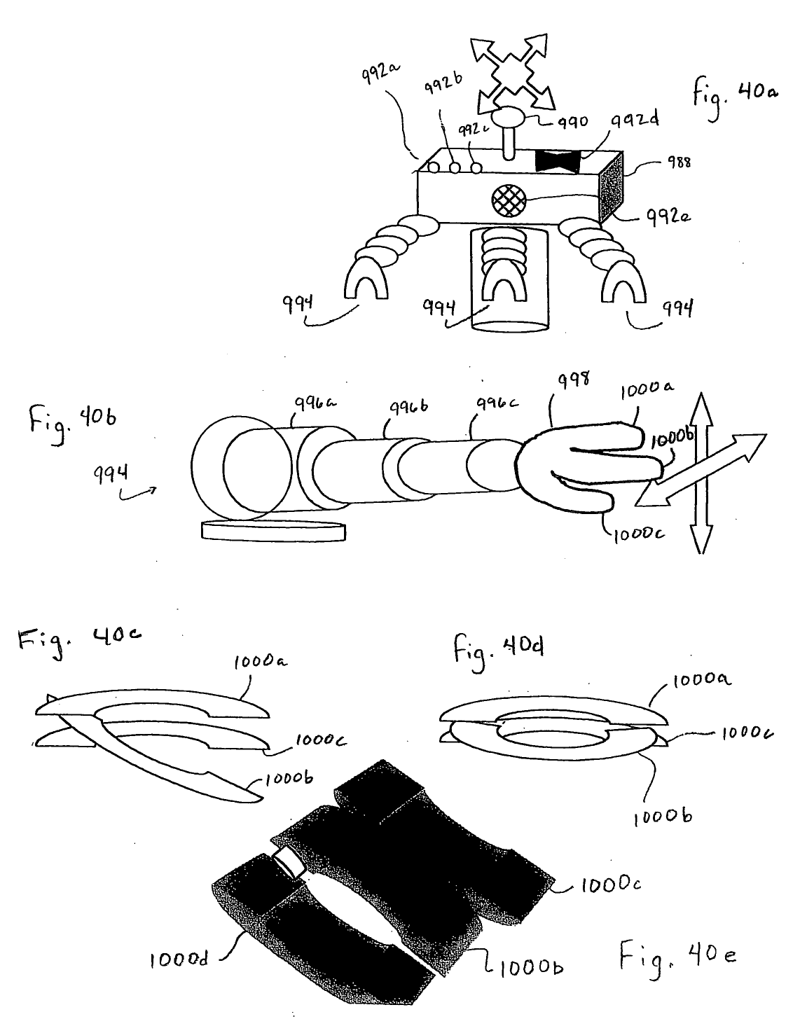

FIG. 40a depicts another embodiment of the present invention. In the embodiment depicted in 40a, a robotic console 988 is provided along with a manual driving stick 990. The manual driving stick 990 operates in conjunction with certain other control features 992a-e. The control features 992a-e may be used to control one or more robotic limbs 994 which are in communication with the robotic console 988. In the embodiment depicted in FIG. 40a, control features 992a, 992b and 992c may be used to pick which robotic limb 994 is currently being manipulated by the robotic console 988. Control feature 992d allows a user to control the height of the robotic console 988 relative to a patient. Control feature 992e is a microphone which may be configured with suitable electronics (including any necessary software in addition to required hardware) to facilitate auditory control of the device.

FIG. 40b depicts a robotic limb 994 for use in accordance with the robotic console 988, such as that depicted in FIG. 40a. The robotic limb 994 may be provided with one or more telescoping sections 996a, 996b and 996c which enable the retraction or extension of the robotic hand 998 attached to the robotic limb 994. Robotic hand 998 may be configured in a similar fashion as robotic tool 974 described in FIG. 39. As described with respect to the tool 974 of FIG. 39, the robotic hand 998 may move in a variety of directions including up and down, laterally right to left or backwards and forwards with further refined movements, including rotation, of one or all of the finger members 1000a, 1000b, and 1000c.

FIG. 40c depicts the finger members 1000a, 1000b, and 1000c of the robotic hand in an open position.

FIG. 40d depicts the finger members 1000a, 1000b and 1000c of the robotic hand in a closed position. Referring to FIGS. 40c and 40d, it may be appreciated how finger members 1000a, 1000b and 1000c may be moved between an open positions, such as that shown in FIG. 40c and a closed position, such as that shown in FIG. 40d, so as to grasp and/or release an object, material or tissue.

FIG. 41 depicts the robotic console 988 as previously described. However, the robotic console 988 is used in conjunction with an operating table 1002. In certain embodiments, the robotic consol 988 may be installed as part of the operating table 1002.

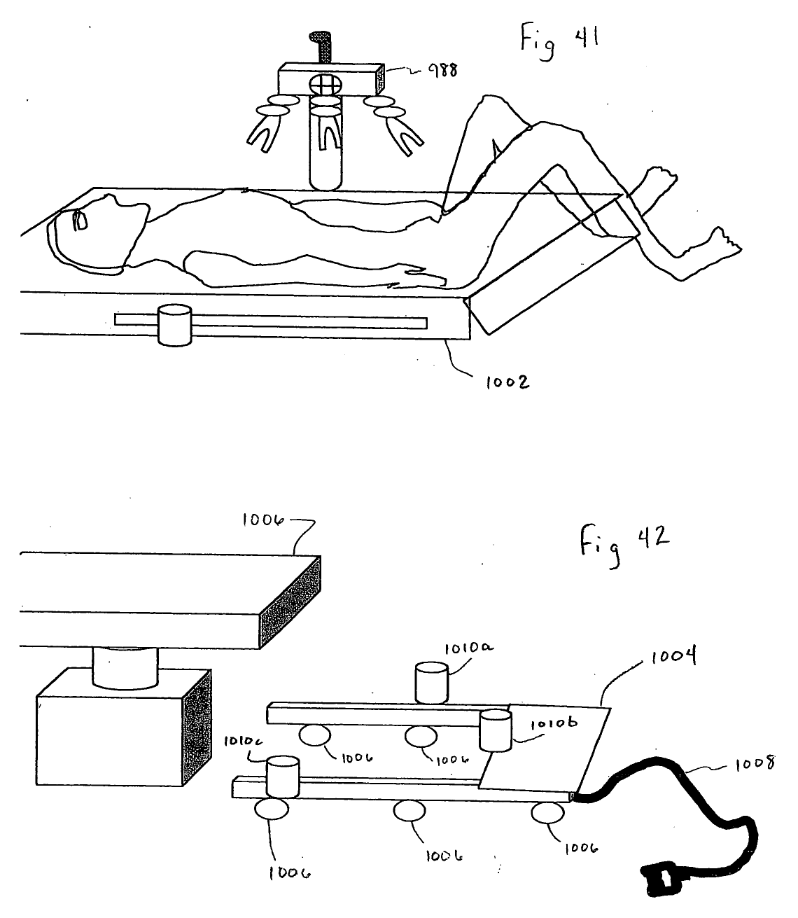

FIG. 42 shows a movable pedestal 1004 in accordance with one embodiment of the invention. The movable pedestal 1004 may be configured with one or more caster wheels 1006 attached thereto for ease of movement in and around the surgical area. A power cord 1008 may also be provided as part of the pedestal 1004. The robotic console 988 may be position at multiple places on the movable pedestal 1004 as shown by mounting features 1010a, 1010b, and 1010b. The movable pedestal 1004 is configured so that multiple robotic consoles 988 may be installed thereupon. The preferable orientation for the multiple robotic consoles 988 is such that one robotic console is provided on either side of the patient and a third robotic console is provided between the legs of a positioned patient.

Alternatively, one or more robotic limbs 994, as shown in FIG. 40b, may be mounted to the movable pedestal 1004. These separately mounted robotic limbs may be controlled by one or more robotic consoles 988, such as that depicted in FIG. 40a. The moveable pedestal 1004 is preferably configured so that it will fit around an operating table 1006.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

What is claimed is:

1. An optical device for minimally invasive medical procedures, comprising:
a plurality of linear image acquisition devices, wherein each of said linear image acquisition devices is adapted to acquire an image from within a patient's body;
a linear housing laterally surrounding said plurality of linear image acquisition devices such that the linear image acquisition devices extend toward an end of the housing, wherein at least a portion of said housing may be inserted within a patient's body and wherein said housing is adapted for cleaning and sterilization;
at least one input adjustment device disposed upon at least one end of the plurality of linear image acquisition devices such that said input adjustment device facilitates adjustment of one or more characteristics of said image; and
a display system, wherein said display system is configured to display the images acquired by the linear image acquisition devices in multiple formats.

2. The optical device of claim 1, wherein said display system is configured to display the images acquired by the linear image acquisition devices in a two-dimensional and a three-dimensional format.

3. The optical device of claim 1, wherein said input adjustment device provides a lens and a reflective surface and wherein said optical device further comprises a protective cover associated with the housing.

4. The optical device of claim 1, wherein said input adjustment device provides a digital manipulator.

5. The optical device of claim 1, further comprising at least one light source.

6. The optical device of claim 1, further comprising a controller for receiving and responding to instructions to control at least one of the display system and the input adjustment device.

7. The optical device of claim 1, wherein said display system provides a single image combining the images from the plurality of linear image acquisition devices.

8. The optical device of claim 1, wherein said display system provides a separate image for two or more of the plurality of linear image acquisition devices.

9. The optical device of claim 1, wherein said display system includes an eyeglasses frame having an image presentation mechanism, said image presentation mechanism presenting a left image to a wearer's left eye and a right image to a wearer's right eye.

10. The optical device of claim 9, wherein at least a portion of said image presentation mechanism may be rotated away from a wearer's eyes, to permit vision beyond the image presentation mechanism without removal of the eyeglasses frame.

11. The optical device of claim 1, wherein one linear image acquisition device provides a left image from a front side of the housing and a second linear image acquisition device provides a right image from a front side of the housing and a third linear image acquisition device is disposed to provide a posterior image from a back side of the housing and wherein said display system provides any of the left image, the right image, and the posterior image as selected by a user.

12. A device for minimally invasive medical procedures, said device comprising:
a hand piece configured for either a right gloved hand of a user, a left gloved hand of a user, or a right or a left gloved hand of a user;
an instrument portion attached to said hand piece, said instrument portion including a tool;
a trigger attached to said hand piece and associated with said instrument wherein said trigger may be manipulated by a finger of a user and said trigger is useful to manipulate said instrument; and
one or more control elements attached to said hand piece and associated with said instrument wherein said one or more control elements may be manipulated by one or more fingers of a user and are useful to manipulate said instrument.

13. The device of claim 12, wherein said hand piece comprises:
an interior portion configured to accept the user's gloved hand; and
an opening to allow access to said interior portion.

14. The device of claim 12, wherein said instrument portion further comprises:
a transcutaneous shaft configured to attach to said hand piece;
an internal shaft extending away from said, said internal shaft being rotatably attached to said transcutaneous shaft and having a knob with a mechanism that causes the internal shaft to rotate in response to manipulation of said knob, wherein said internal shaft is detachable and wherein said tool is detachable to said internal shaft.
a tool affixed to an end of the internal shaft, wherein said tool is detachably affixed to said internal shaft.

15. The device of claim 12, wherein said tool is selected from the group consisting of forceps, flat scissors, curved scissors, right angle scissors, DeBakey-type forceps, right angle forceps, blunt forceps, curved clamps, angular clamps, ultrasound probes, lasers, cautery devices, staplers, circular staplers, knives, suturing devices, rivet drivers, ligation devices, aspiration devices, injection devices, biopsy devices, radiotherapy devices; and radioactive emitter loading devices, wherein manipulation of one or more of said one or more control elements causes operation of said tool and wherein manipulation of said trigger causes said tool to rotate to a new position.

16. A device in accordance with claim 15, wherein said tool is a stapler configured to staple tissue together by providing two pairs of overlapping rows of staples, one pair on either side of a cut line and wherein said stapler provides two lines of tissue compression, one line of tissue compression being on the outer side of each of the pairs of overlapping rows of staples.

17. The device of claim 12 further comprising:
at least one sensory pad associated with one or more of said control elements, said sensory pad being configured to transmit a pressure signal from a user's finger; and
a mechanism to operate said tool, wherein the mechanism to operate said tool is responsive to said pressure signal.

18. The device of claim 12 further comprising:
a mechanism to operate said tool, wherein said mechanism to operate said tool is configured to transmit feedback to the user, said feedback relating to the operation of said tool.

19. The device of claim 12 further comprising:
a temperature button, wherein said temperature button is attached to said hand piece and is configured to generate a signal upon manipulation by a user, wherein said tool is responsive to said signal generated by said temperature button so as to cause at least a portion of said tool to heat up so as to be useful to cauterize tissue.

20. The device of claim 12, wherein said instrument portion is configured to detachably engage a variety of replaceable tools and wherein said device further comprises a replaceable tool.

21. The device of claim 12, further comprising a band which secures the device to the wrist, or palm, or both the wrist and palm of a user.

22. A device for minimally invasive medical procedures, said device comprising:
a scissor-type hand piece; said hand piece having a first elongated portion and a second elongated portion, said first elongated portion being adapted for manipulation by a user's thumb and said second elongated portion being adapted for manipulation by one or more of user's first, second, third, and fourth fingers;
a temperature control element attached to said hand piece and configured to generate a signal upon manipulation by a user's finger; and
an instrument portion attached to said hand piece wherein said instrument portion is responsive to manipulation of the first elongated portion and second elongated portion, wherein said instrument portion includes a tool, said tool being responsive to said signal generated by said temperature control element so as to cause at least a portion of said tool to heat up so as to be useful to cauterize tissue, said tool being detachable.

23. A tool for use with a device for minimally invasive medical procedures, said tool comprising:
an elongated first element, an elongated second element and an elongated third element; wherein said first element and said second element are opposed to said third element and said first element is configured to mimic the functionality of a first finger of a user, and said second element is configured to mimic the functionality of a second finger of a user, and said third element is configured to mimic the functionality of a thumb of a user, wherein at least one of said first element, second element, and third element includes a sensory pad configured to transmit a pressure sensation from said element to a user's finger.

24. An automated device for minimally invasive medical procedures, said device comprising:
a robotic console;
a plurality of control features; and
one or more robotic limbs.

25. The automated device of claim 24, said device further comprising:
a mobile pedestal, wherein said one or more robotic limbs are affixed to said mobile pedestal.
